(19) United States
(12) Patent Application Publication
(10) Pub. No.: US 2026/0318911 A1
(43) Pub. Date: Oct. 15, 2026

(54) SALIVARY ENDOTOXIN DETECTION USING COMBINED MONO/POLYCLONAL ANTIBODY-BASED SANDWICH-TYPE LATERAL FLOW IMMUNOASSAY DEVICE

(71) Applicant: University of Cincinnati, Cincinnati, OH (US)

(72) Inventors: Andrew J. Steckl, Cincinnati, OH (US); Daewoo Han, Cincinnati, OH (US); Sancai Xe, Cincinnati, OH (US)

(21) Appl. No.: 19/642,839

(22) Filed: Apr. 9, 2026

Related U.S. Application Data
(60) Provisional application No. 63/785,704, filed on Apr. 9, 2025.

(51) Int. Cl. G01N 33/543; B82Y 15/00; G01N 21/65; G01N 33/569

(52) U.S. Cl. CPC G01N 33/54388; G01N 21/658; G01N 33/54346; G01N 33/56911; B82Y 15/00; G01N 2400/50

(57) ABSTRACT

A lateral flow assay device for detecting the presence of, or quantifying an amount or concentration of, analyte in a sample. The device includes (1) a conjugate region including a binding reagent coupled to a signal-generating particle, wherein the binding reagent comprises a first antibody configured to bind lipopolysaccharide, and wherein the binding reagent is mobile relative to the device, and (2) an analytical membrane in fluid communication with the conjugate region, the analytical membrane comprising a test region and a control region. The test region includes an immobilized capture reagent configured to capture a complex including the analyte and the binding reagent, wherein the immobilized capture reagent is a second antibody. The control region includes a control component configured to capture the binding reagent coupled to the signal-generating particle.

### CROSS-REFERENCE TO RELATED APPLICATIONS

This application claims priority to, and the benefit of the filing date of, U.S. Provisional Patent Application No. 63/785,705, filed on Apr. 9, 2025, and titled “Salivary Endotoxin Detection Using Combined Mono/Polyclonal Antibody-Based Sandwich-Type Lateral Flow Immunoassay Device, the disclosure of which is incorporated by reference herein in its entirety.

### FIELD OF THE INVENTION

This invention relates generally to lateral flow immunoassay devices.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Biological fluids, such as blood, urine, saliva, and mucus are maybe the most important sources of information regarding our medical status. Historically, and still the case today, blood analysis is the most common tool of diagnosis. Conventional detection of molecular markers found in biofluid specimens is carried out mostly utilizing the well-known polymerase chain reaction (PCR) method, which requires trained personnel and a relatively longer time span in a laboratory setting [1]. However, with the ever-growing use of point-of-care (POC) diagnostics, other biofluids are gaining in importance. Rapid and low-cost analysis of biomarkers present in various biofluids can provide early indication of various medical conditions, thus improving treatment outcomes while reducing medical costs [2]. As demonstrated during the coronavirus disease (COVID) pandemic, lateral flow assay (LFA) is a versatile POC platform with many benefits, such as fast analysis time, low cost, user-friendly colorimetric interface, simple fabrication process, and no need for external instruments [3]. Because COVID LFA-based home test kits were widely utilized during the pandemic, a world-wide familiarity with LFA test kits now exists. Furthermore, the use of smartphones has been developed for LFA applications not only to enhance the analysis of test results but also to improve the LFA sensitivity [4].

One of the most important considerations in LFA development for POC use is the type and nature of sample fluid. Saliva is an attractive biofluid for POC applications, because of stress-free non-invasive sampling and excellent availability in significant sample volume. Saliva is also a versatile medium for analysis as it consists of a wide mixture of proteins, peptides, DNA, cell debris, and food particles that contains markers associated with a variety of medical conditions including high stress levels, bacterial and viral infections, presence of drugs and other toxic compounds, etc. [3,5]. However, this also presents challenges in isolating the specific biomarker of interest for analysis [6].

Bacterial endotoxins found in saliva contain lipopolysaccharides (LPS), important macromolecules existing in the outermost part of Gram-negative bacteria that are released after cell death and lysis. Endotoxins mainly consist of lipid and polysaccharide parts as shown in FIG. 1A. Lipid A is the most toxic part, and the polysaccharide has diverse configurations depending on different species and strains of the bacteria [7]. LPS is a complex heterogeneous biomacromolecule. Its effective molecular weight can range from 10,000 to several million Daltons because it normally exists as an agglomeration (e.g. micelles and vesicles) in aqueous solution [8]. A large number of LPS molecules are released when bacteria die or are metabolically stressed. For example, a single E. coli bacterium can release 2-3 million LPS molecules after death [9,10]. Once LPS is released, the toxic lipid A part is exposed to the host's immune system. LPS elicits strong defense reactions, such as immune responses, various inflammations, life-threatening sepsis, and possibly septic shock [11].

Endotoxins induce a strong immune response in hosts but also serve as important biomarkers useful for diagnosing various bacterial diseases. Porphyromonas gingivalis (P. gingivalis), one of more than 700 bacterial species found in the oral cavity, and one of 500 species present in subgingival plaque [12] is a major Gram-negative pathogenic bacterium that is responsible for chronic periodontitis. Periodontal disease presents a chronic inflammation caused by bacterial infection, leading to gum disease and even loss of teeth. LPS is increased in subgingival plaques in gingivitis sites as quantified using TLR4 reporter gene assays and Lumulus Amebocyte Lysate (LAL) test [13,14]. LPS from P. gingivalis (PG LPS) induces significant host responses in gingival tissue by increasing the production of inflammatory biomarkers, such as interleukin (IL)-1β, IL-6, IL-8, interferon-γ and tumor necrosis factor alpha (TNF-α) in gingival tissue [15]. The salivary presence of this bacteria in a high concentration suggests that the patient has a chronic periodontitis disease. Furthermore, because of the close relationship between P. gingivalis and other important systemic diseases, such as cardiovascular [16, 17], rheumatoid arthritis [18], and neurodegenerative (Alzheimer's) diseases [19-21] that has emerged in recent years, quantitative evaluation of P. gingivalis LPS has become a key measure in oral health, as well as in the whole body health system [1,22,23]. LAL tests have been utilized as a gold-standard for LPS detection, mainly of the lipid A part. Other methods such as enzyme-linked immunosorbent assays (ELISA) [25], electrochemical (EC) sensing [26], electrochemical impedance spectroscopy (EIS) [27], have been developed [28]. However, they have various challenges, such as specificity and portability for POC diagnostics.

Further, while LFAs have become ubiquitous for the rapid point-of-care diagnosis of multiple conditions ranging from pregnancy to infections to high stress levels, while utilizing a variety of biofluids (saliva, urine, sweat, etc.) [29-33], conventional colorimetric LFA diagnoses are only possible with relatively high biomarker concentrations [34]. This issue was well-documented during the COVID pandemic when LFA tests were not able to detect pre-symptomatic infections, due to lower viral loads [35]. Conversely, sensitive diagnostic methods, such as ELISA or PCR analysis, require sample collection and off-site analysis in specialized labs, resulting in a time lapse between sampling and results. Thus, there is a demand for developing testing methods that have sensitivity comparable to lab-based molecular tests while executed at the point-of-care sites. Without filling this technological gap, surveillance and diagnostic infrastructure will remain vulnerable to the spread of infectious diseases.

To this end, the standard colorimetric LFA protocol has been expanded by modifying its sensing mechanism and/or readout method. Expansion of the standard LFA protocol has produced a number of techniques, including target amplification, catalytic colorimetric response, thermal response, use of smart phone image detection and processing and surface-enhanced Raman (SERS) coupled LFAs [37-40]. An LFA with non-covalent DNA catalytic amplification has been demonstrated for detection of SARS-COV-2 with picomolar detection limits [29]. These devices, dubbed PLAN-LFA, employ a padlock rolling circle amplification to increase sensitivity 1000 times, however the assays, which take approximately three hours, are still not rapid enough for in-office diagnosis. A catalytic colorimetric response was developed by replacing conventional gold nanoparticles (AuNP) with palladium nanoparticles (Pd NPs), which function as a catalyst for color-changing reporter dye. The presence of a catalyst amplifies the signal observed for each NP without adding significant processing time (incubation <20 min) [40]. Thermal contrast has been used to improve sensitivity eight-fold over standard optical detection [38]. In this application, an IR camera measures the temperature at the test and control lines under laser irradiation, due to the strong plasmonic photothermal effect generated by NPs. This strategy improves sensitivity by changing the readout method without modifying the LFA. While these methods are powerful ways of increasing the sensitivity of LFA, they require complicated protocols and long testing times under the controlled environment, resulting in a low accessibility for users.

The SERS-based LFA (SERS-LFA) diagnostic method can exploit the advantages given by the vibrational nature of spectroscopic readout, which permits for molecular-level sensitivity, highly multiplexed analysis, and easy signal extraction from broad backgrounds. SERS can be a user-friendly method because it simply adds the Raman reporter on metal nanoparticles used for conventional LFA, while using the same testing procedure. After the first demonstration of LFA using SERS detection in 2017, SERS-LFAs have been explored extensively and summarized well in the literature [39, 42-55]. These studies have demonstrated increased sensitivity and multiplexed analysis enabled by the combination of LFA with a SERS readout [56, 57]. Studies have initially incorporated a SERS active Raman reporter onto standard AuNP [47, 49, 50] and introduced different nanomaterials (e.g. nanostar) to further increase the sensitivity with various properties [44-46, 48, 51-55]. Hwang et al. demonstrated the first SERS-LFA biosensor that presented three orders of magnitude higher sensitivity than the corresponding ELISA-based method [41]. The capability of simultaneous multiplexing detection using SERS-LFA were also demonstrated for detection of dual nucleic acids and two different diseases such as Zika and Dengue [59]. Instead of spherical AuNP, gold nanostars with a branched structure were utilized to detect bisphenol A using SERS-LFA, presenting 20 times higher sensitivity than that of colorimetric quantification [54]. However, only a few reports have compared the performance of different nanomaterials to optimize the LFA itself [54]. To systematically improve the capabilities of LFAs, side-by-side comparison and associated optimization are necessary. More recently, to improve user accessibility, portable Raman systems have been demonstrated for measurement of SERS-LFAs [56, 60, 61]. This affordable and easy-to-use portable system enables SERS-LFAs to be used for POC applications.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

As described above in the Background section, challenges currently exist with providing a lateral flow immunoassay test strip that is rapid and sensitive enough that it can be used in point-of-care type applications.

With that in mind, the present inventors have developed a point-of-care lateral flow assay (LFA) device to detect and quantify PG LPS concentrations in human saliva using the molecular sandwich capture approach. Since LPS is a large biomacromolecule with multiple binding sites, one can beneficially utilize the more sensitive sandwich-type immunoassay rather than the competitive approach [62]. The size of LPS aggregates in buffer solutions were evaluated using dynamic light scattering (DLS) method. Interestingly, there is no significant difference in size with relatively tight distribution considering low PDI values.

The present inventors have evaluated two different designs of sandwich-based LFA devices. In the first design (FIG. 1B), two different antibodies are utilized in the device. A monoclonal antibody (mAb) is conjugated to the surface of gold nanoparticles (AuNP) to capture target PG LPS molecules in the sample solution. A polyclonal antibody (pAb) is printed and immobilized on the nitrocellulose (NC) membrane of the LFA device forming a test line that captures the AuNP conjugates bound to PG LPS from the sample solution. When LPS is present in the sample solution, the mAb-conjugated AuNP particles (mAb-AuNP) bind to LPS. Then the combined LPS-mAb-AuNP conjugate particles flow through the porous NC membrane and are captured by the pAb immobilized in the test line stripe on the LFA. Polyclonal antibodies can bind to other available binding sites on the LPS molecules or LPS micelles not occupied by the mAb-AuNP. When sufficient LPS-mAb-AuNP particles are captured a reddish test line is observed on the NC membrane. The control line on the LFA is printed on the NC membrane with protein G, which captures the mAb conjugated on the AuNP. This validates the test by confirming the conjugation status between mAb and AuNP. However, this design is not ideal for POC applications because it needs a relatively long (40 min) incubation time to bind mAb-AuNP conjugates to the target LPS in sample solution. This limitation can be resolved when conjugated Ab-AuNP are pre-loaded into the conjugation pad.

In the second approach (FIG. 1C) the polyclonal antibody is utilized both for AuNP conjugation and the test line immobilization. When the sample solution is dispensed on the sample pad and proceeds to hydrate the conjugation pad, the immobilized pAb on AuNP surface quickly binds to the PG LPS in the sample solution. The pAb-conjugated AuNPs are released from the conjugation pad and flow through the NC membrane. In the presence of LPS in the sample, the formed AuNP-pAb-LPS conjugates are captured by the immobilized pAb on the test line. A fraction of the AuNP-pAb that do not bind to LPS are captured by protein G immobilized on the control line. Interestingly, although the same antibody was utilized for both AuNP conjugation and immobilization on the test line, the sandwich structure was successfully formed due to the multiple binding sites of available on the polyclonal antibodies. Apparently, this approach does not require the incubation time to bind the pAb-AuNP conjugates to PG LPS in the sample solution, leading to faster LFA test results.

And so, one aspect of the present invention is directed to a lateral flow assay device for detecting the presence of, or quantifying an amount or concentration of, lipopolysaccharide in a sample. The device in this aspect of the invention can be used in point-of-care applications. The device of this aspect of the invention, in one embodiment, includes (1) a conjugate region including a binding reagent coupled to a signal-generating particle, wherein the binding reagent comprises a monoclonal antibody configured to bind lipopolysaccharide, and wherein the binding reagent is mobile relative to the device; and (2) an analytical membrane in fluid communication with the conjugate region. The analytical membrane includes a test region and a control region. The test region includes an immobilized capture reagent configured to capture a complex including the lipopolysaccharide and the binding reagent, wherein the immobilized capture reagent comprises a polyclonal antibody. And the control region includes a control component configured to capture the binding reagent coupled to the signal-generating particle. The device may also include a sample receiving region in fluid communication with the conjugate region, and an absorbent region in fluid communication with the analytical membrane.

Another embodiment of this aspect of the invention is a lateral flow assay device for detecting the presence of, or quantifying an amount or concentration of, lipopolysaccharide in a sample. The device of this embodiment includes (1) a conjugate region including a binding reagent coupled to a signal-generating particle, wherein the binding reagent comprises a first polyclonal antibody configured to bind lipopolysaccharide, and wherein the binding reagent is mobile relative to the device; and (2) an analytical membrane in fluid communication with the conjugate region. The analytical membrane includes a test region and a control region. The test region includes an immobilized capture reagent configured to capture a complex including the lipopolysaccharide and the binding reagent, wherein the immobilized capture reagent comprises a second polyclonal antibody. The control region comprises a control component configured to capture the binding reagent coupled to the signal-generating particle. The device may also include a sample receiving region in fluid communication with the conjugate region, and an absorbent region in fluid communication with the analytical membrane.

Further, as described above in the Background section, the introduction of surface-enhanced Raman scattering (SERS) in lateral flow assays (LFA) has been recently exploited to increase the sensitivity and quantification capabilities of these rapid tests. However, there are still significant drawbacks to SERS-LFAs currently being used and researched, such as in the particular nanomaterials being currently used in such LFAs. Herein, the present inventors took advantage of a SERS-LFA combination to improve LFAs designed to test for P. gingivalis-a biomarker for oral health. As noted above, endotoxin released from a Gram-negative pathogenic P. gingivalis is causing chronic inflammation in the gum tissue and leading to tooth loss if not treated in time. In addition, some important cardiovascular and neurodegenerative diseases recently revealed a very close relationship to P. gingivalis concentration in oral cavity [63, 64]. Therefore, it becomes more important to monitor the PG LPS level for oral health as well as whole body health using a POC monitoring capability.

Previous work has reported the development of P. gingivalis colorimetric LFA detecting lipopolysaccharides (LPS) produced by the bacteria with a limit of detection (LOD) of ~22 ng/ml and a dynamic range up to ~10-20 μg/mL [31]. To utilize the LFA as a diagnostic test for early onset of oral diseases it is important to reduce the LOD below 10 ng/mL. Clinically, the average LPS concentrations of healthy individuals and periodontitis patients are approximately 10 ng/ml and 31 ng/ml, respectively [65].

Here the present inventors report on the combination of this LFA for P. gingivalis with a SERS readout with the LOD <10 ng/ml, which is within the range of concentration needed to monitor oral health. By comparison, conventional colorimetric LFAs achieve an LOD≈100 ng/mL, generating a lower diagnostic sensitivity. To achieve this enhanced sensitivity, the present inventors optimized the materials used in the SERS-LFA, investigating nanostars (NS) differing in size and material composition and comparing them to commercial gold nanoparticles, as a benchmark. More specifically, the present inventors explored five different metallic nanomaterials to determine the optimal nanomaterial for incorporation into SERS-LFAs: gold spheres/nanoparticles (AuNP), two sizes each of gold nanostars (AuNS) and silver-coated gold nanostars (Ag@AuNS). The Ag@AuNS has been shown to have superior single-particle signal enhancement although this property has not been tested in LFA systems yet. In particular, present inventors found that large (56-nm) NSs with a silver coating were the most sensitive nanomaterials for SERS-LFA.

To prove the applicability of this SERS-LFA to point-of-care (POC) settings, the present inventors tested the optimized LFA with a portable Raman system prototype designed to work on LFAs with 3D-printed cartridges and a line-shaped laser illumination. This work represents the first analytical comparison of more than two materials for optimization of SERS-LFAs. The different nanomaterials were first characterized and functionalized with a Raman reporter and the SERS enhancement of each nanomaterial was evaluated when collected at an LFA control line formed without the target PG LPS. Then, using the optimized nanomaterial, the SERS-LFA was calibrated for the detection of LPS produced by P. gingivalis and the sensitivity was compared to that of a colorimetric LFA. Using this prototype, the present inventors achieved the same LOD observed with the traditional benchtop Raman system. Thus, the portable Raman system described herein brings the SERS-LFA technology into the arena of POC use.

And so, another aspect of the present invention is directed to a lateral flow assay device for determining the presence of, or quantifying an amount or concentration of, lipopolysaccharide in a sample. An embodiment of a device of this aspect includes (1) a conjugate region comprising a binding reagent coupled to a SERS-active particle and associated with a Raman reporter, wherein the binding reagent is configured to bind lipopolysaccharide, and wherein the SERS-active particle comprises a silver-coated gold nanostar; and (2) an analytical membrane in fluid communication with the conjugate region. The analytical membrane includes a test region and a control region. The test region includes an immobilized capture reagent configured to capture a complex comprising the lipopolysaccharide and the binding reagent. And the control region comprises a control component configured to capture the binding reagent coupled to the SERS-active particle. The device is configured to generate a Raman-detectable signal at the test region.

For the various embodiments described herein, the sample receiving region may be referred to herein as a sample pad; the conjugate region may be referred to herein as a conjugate pad; and the absorbent region may be referred to herein as a wicking pad. The analytical membrane may be a nitrocellulose membrane. These components may be arranged in overlapping configuration in certain embodiments in the following order: sample receiving region, conjugate region, analytical membrane, absorbent region. A sample placed on the sample receiving region (such as in liquid form) may move, flow, migrate, be transported, etc. along the device from sample receiving region toward the absorbent pad (such as by capillary action).

### BRIEF DESCRIPTION OF THE DRAWINGS

The objects and advantages of the disclosed invention will be further appreciated in light of the following detailed descriptions and drawings in which:

FIGS. 1A-1C include schematics showing basic mechanisms and assay designs for combined mono/polyclonal antibody-based lipopolysaccharides (LPS) detection and LFA implementation: FIG. 1A shows structure of LPS released from the Gram-negative bacteria; FIG. 1B shows separate premixing of sample and Ab-AuNP solutions (photo taken after the test); and

FIG. 1C shows simplified approach using polyclonal Ab-AuNPs preloaded on the conjugate pad (photo taken before the test).

FIGS. 2A-2E show optimization of test line formation on LFA devices: FIG. 2A shows non-specific binding removal by adding NaCl solution; FIG. 2B shows incubation time after mixing mAb-AuNP and LPS solutions; FIG. 2C shows the effect of immobilized polyclonal antibody concentrations from 0.1 to 1.0 mg mL⁻¹ on test line intensity on LFA device; FIG. 2D shows change of test line intensity over time at 4 different pAb concentrations; and FIG. 2E shows averaged test line intensities after 10 min (n=3). All images were taken at 10 min after dispensing sample solutions.

FIGS. 3A-3C show LFA test line intensity for detection of P. gingivalis LPS: FIG. 3A shows quantitative LFA results with different LPS concentrations (μg mL⁻¹); FIG. 3B shows lower concentration regime, below 2 μg mL⁻¹; and FIG. 3C shows high concentration regime, up to 20 μg mL⁻¹. (n=3).

FIG. 4 shows data from a selectivity test with mucin and different endotoxins: E. coli (EC), P. pallens (PP), and P. gingivalis (PG). (n=3)

FIGS. 5A-5D show saliva pre-treatments for P. gingivalis LPS LFA tests: FIG. 5A shows the effect of major salivary proteins such as α-amylase and mucin on P. gingivalis LPS detection in LFA tests; FIG. 5B shows saliva treated either with potato starch (top) or potato starch followed by 0.45 μm syringe filtration (bottom); FIG. 5C shows amylase assay results either after incubating with potato starch (left) or one-step syringe filtration with potato starch (right); FIG. 5D shows LFA images with saliva after various pretreatments (SF-only: syringe filtration only; PT-only: potato starch incubation only; SF w. PT: syringe filtration with potato starch). For all samples, LPS was spiked at 2 μg mL⁻¹ concentration.

FIGS. 6A-6C show treated saliva tests with several LPS concentrations: FIG. 6A shows LFA test images of different LPS concentration (μg mL⁻¹) shown in each LFA strip; FIG. 6B shows quantitative analysis using ImageJ (n=3); and FIG. 6C shows selectivity evaluation against various endotoxins.

FIG. 7 includes an image of benchtop Raman setup for measurements on LFA, including schematics of scanning measurements on LFA.

FIG. 8 is a schematic representation of process for colorimetric analysis of LFA strip.

FIG. 9 is a depiction of the research workflow. LFA for P. gingivalis LPS were modified and optimized for SERS readout using several nanomaterials: (top) conventional LFA configuration and sensing mechanism, (bottom) comparison of different plasmonic nanomaterials for SERS and its application to LFA device.

FIGS. 10A and 10B include a series of photographs and graphs showing nanoparticle characterization. (left-to-right) TEM images, absorbance spectra, nanoparticle tracking analysis (NTA), and SERS spectra of (top-to-bottom) AuNP, AuNS, and Ag@AuNS. TEM images shown are 150k× magnification and scale bars are 100 nm. NTA analysis of AuNP and AuNS were performed after coating with thiol-PEG to stabilize the metal surface. Background-subtracted SERS spectra showing the peak of interest (546 cm⁻¹) for the reporter, used to determine SERS intensity.

FIGS. 11A-11E includes a series of graphs showing calibration of OD as a function of concentration (measured by NTA) for the different nanomaterials used to compare material SERS intensities normalized for concentration for AuNP (FIG. 11A), Au NS 27 nm (FIG. 11B), Au NS 40 nm (FIG. 11C), Ag@Au NS 39 nm (FIG. 11D), and Ag@Au NS 56 nm (FIG. 11E).

FIGS. 12A-12C show analysis of nanoparticle performance in LFA strips: FIG. 12A shows images of LF As with only visible control line; FIG. 12B shows that each LFA was measured with the LFA scanner device and the reporter signal intensities for single 1-s acquisitions, the increased signal at point 21 corresponds to the control line; FIG. 12C shows SERS spectra accumulated from three horizontal (90° with respect to the flow direction) scans (n=30 spectra) collected across the control line. The total scan time for a full length of LFA and region-of-interest at the test and/or control lines is ~3 min.

FIGS. 13A-13E include intensity maps associated with the spectral data shown in FIG. 12C, showing the variance in intensity across the control line.

FIGS. 14A-14F show the characterization of SERS-LFA with increasing concentrations of LPS. SERS with 56 nm Ag@AuNS: FIG. 14A includes LFA photos; FIG. 14B includes SERS intensity maps (6×30-point parallel line-scans performed over the strips); FIG. 14C shows average line-scan at increasing LPS concentrations; FIG. 14D shows SERS intensity from the test line (point #10 in FIG. 14C) as a function of LPS concentration, showing data points from single LFA sets and LOD level (error bars represent standard deviation from 3 different LFAs). Characterization of AuNP colorimetric LFA with increasing concentrations of LPS: FIG. 14E includes LFA photos; FIG. 14F optical intensity from test line as a function of LPS concentration, showing data points from single LFA sets and LOD level (error bars represent standard deviation from 3 different LFAs). LOD level was calculated as the blank signal+3×standard deviation of the blank. All SERS intensities are reported for the 546 cm⁻¹ peak of interest.

FIGS. 15A and 15B are graphs, with FIG. 15A showing a scaled version of FIG. 14C showing the target line intensities for blank, 1, 10, 100 ng/ml (not easily visible in the original figure); and FIG. 15B showing spectra for the target line for blank, 1, 10 ng/ml with a reference spectrum for the specific Raman reporter, showing single spectra for each measurement (light line) and their average (thick line).

FIGS. 16A-16E show a comparison of SERS-LFA performance with benchtop and portable Raman systems. FIG. 16A is a photo of SERS-LFA device prototype. Spectra and SERS signal (@546 cm⁻¹) from a set of LFAs run with different concentrations of LPS for benchtop (FIGS. 16B and 16C) and portable Raman system (FIGS. 16D and 16E). In the SERS spectra both the average spectrum and each measurement spectrum are reported (n=6 and 9 for FIGS. 16B and 16D, respectively). The single measurement spectra are reported with a lighter shade of the corresponding color. Error bars represent the standard deviation over 6 line-scans for the benchtop instrument and 9 independent measurements (spectra) for the portable. LOD level was calculated as the blank signal+3×standard deviation of the blank.

FIGS. 17A and 17B are spectra of control lines associated with the strip measured in FIGS. 16A-16E, for benchtop (FIG. 17A) and portable Raman (FIG. 17B). Single spectra for each measurement are reported with light lines and their average as a thick line.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As described above in the Background section, challenges currently exist with providing a lateral flow immunoassay test strip that is rapid and sensitive enough that it can be used in point-of-care type applications.

With that in mind, the present inventors have developed a point-of-care lateral flow assay (LFA) device to detect and quantify PG LPS concentrations in human saliva using the molecular sandwich capture approach. Since LPS is a large biomacromolecule with multiple binding sites, one can beneficially utilize the more sensitive sandwich-type immunoassay rather than the competitive approach [62]. The size of LPS aggregates in buffer solutions were evaluated using dynamic light scattering (DLS) method. Interestingly, there is no significant difference in size with relatively tight distribution considering low PDI values.

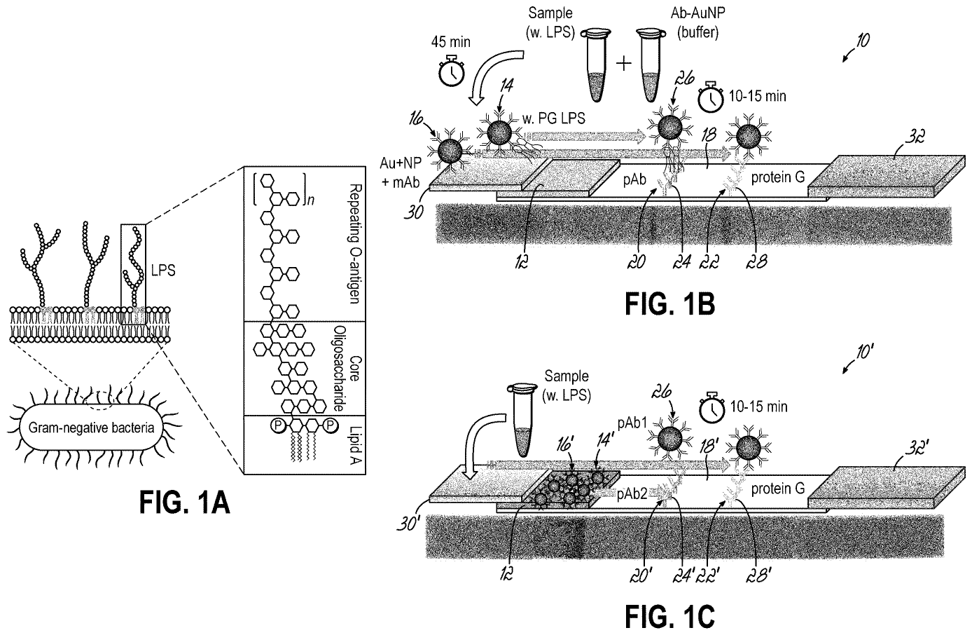

The present inventors have evaluated two different designs of sandwich-based LFA devices. In the first design (FIG. 1B), two different antibodies are utilized in the device. A monoclonal antibody (mAb) is conjugated to the surface of gold nanoparticles (AuNP) to capture target PG LPS molecules in the sample solution. A polyclonal antibody (pAb) is printed and immobilized on the nitrocellulose (NC) membrane of the LFA device forming a test line that captures the AuNP conjugates bound to PG LPS from the sample solution. When LPS is present in the sample solution, the mAb-conjugated AuNP particles (mAb-AuNP) bind to LPS. Then the combined LPS-mAb-AuNP conjugate particles flow through the porous NC membrane and are captured by the pAb immobilized in the test line stripe on the LFA. Polyclonal antibodies can bind to other available binding sites on the LPS molecules or LPS micelles not occupied by the mAb-AuNP. When sufficient LPS-mAb-AuNP particles are captured a reddish test line is observed on the NC membrane. The control line on the LFA is printed on the NC membrane with protein G, which captures the mAb conjugated on the AuNP. This validates the test by confirming the conjugation status between mAb and AuNP. However, this design is not ideal for POC applications because it needs a relatively long (40 min) incubation time to bind mAb-AuNP conjugates to the target LPS in sample solution. This limitation can be resolved when conjugated Ab-AuNP are pre-loaded into the conjugation pad.

In the second approach (FIG. 1C) the polyclonal antibody is utilized both for AuNP conjugation and the test line immobilization. When the sample solution is dispensed on the sample pad and proceeds to hydrate the conjugation pad, the immobilized pAb on AuNP surface quickly binds to the PG LPS in the sample solution. The pAb-conjugated AuNPs are released from the conjugation pad and flow through the NC membrane. In the presence of LPS in the sample, the formed AuNP-pAb-LPS conjugates are captured by the immobilized pAb on the test line. A fraction of the AuNP-pAb that do not bind to LPS are captured by protein G immobilized on the control line. Interestingly, although the same antibody was utilized for both AuNP conjugation and immobilization on the test line, the sandwich structure was successfully formed due to the multiple binding sites of available on the polyclonal antibodies. Apparently, this approach does not require the incubation time to bind the pAb-AuNP conjugates to PG LPS in the sample solution, leading to faster LFA test results.

As noted above, the various aspects of the present invention are directed to lateral flow assay devices. Such devices generally may include a plurality of components arranged so that a sample (such as a liquid sample) can migrate move through the device. In certain embodiments, the device can include a sample receiving region, a conjugate region, an analytical membrane, and an absorbent region.

For the various embodiments described herein, the sample receiving region may be referred to herein as a sample pad; the conjugate region may be referred to herein as a conjugate pad; and the absorbent region may be referred to herein as a wicking pad. The analytical membrane may be a nitrocellulose membrane. These components may be arranged in overlapping configuration in certain embodiments in the following order: sample receiving region, conjugate region, analytical membrane, absorbent region. A sample placed on the sample receiving region (such as in liquid form) may move, flow, migrate, be transported, etc. along the device from sample receiving region toward the absorbent pad (such as by capillary action). Further, the device of the various embodiments described herein may be provided as an exposed strip or enclosed within a cassette, cartridge, or other housing. In some embodiments, the housing includes a sample application opening and a window aligned with the analytical membrane, such as for viewing the test region and/or control region.

In certain embodiments, the device may include a backing support. The backing support may be substantially non-porous and may provide structural support and alignment for one or more of the various components of the device, such as the sample receiving region, the conjugate region, the analytical membrane, and the absorbent region. Materials for backing supports are generally known, and can include polymer films and laminates, including polyester, polyvinyl chloride, polycarbonate, polystyrene, and related materials. The backing support may include an adhesive layer to secure one or more of the components of the device (e.g., the sample receiving region, the conjugate region, the analytical membrane, and the absorbent region) to the backing support.

And so, one aspect of the present invention is directed to a lateral flow assay device for detecting the presence of, or quantifying an amount or concentration of, lipopolysaccharide in a sample. The device in this aspect of the invention can be used in point-of-care applications. The device 10 of this aspect of the invention, in a first embodiment (and referring to FIG. 1B), includes (1) a conjugate region 12 including a binding reagent 14 coupled to a signal-generating particle 16, wherein the binding reagent 14 comprises a monoclonal antibody configured to bind lipopolysaccharide, and wherein the binding reagent 14 is mobile relative to the device 10; and (2) an analytical membrane 18 in fluid communication with the conjugate region 12. The analytical membrane 18 includes a test region 20 and a control region 22. The test region 20 includes an immobilized capture reagent 24 configured to capture a complex 26 including the lipopolysaccharide and the binding reagent 14, wherein the immobilized capture reagent 24 comprises a polyclonal antibody. And the control region 22 includes a control component 28 configured to capture the binding reagent 14 coupled to the signal-generating particle 16. The device 10 may also include a sample receiving region 30 in fluid communication with the conjugate region 12, and an absorbent region 32 in fluid communication with the analytical membrane 18.

In this first embodiment, the sample receiving region 30 is configured to receive the sample to be analyzed for the presence or quantity of a particular analyte or analytes. The sample receiving region 30 may be a porous sample pad, and may comprise materials including, but not limited to, cellulose, glass fiber, rayon, polyester, a woven material, and a nonwoven material. The sample receiving region 30 may optionally include one or more agents configured to improve performance of the device 10 (e.g., buffer, surfactant, preservative, etc.)

The conjugate region 12 is in fluid communication with the sample receiving region 30 and contains a binding reagent 14 configured to bind analyte (such as lipopolysaccharide). In this first embodiment (and in others), the binding reagent 14 is coupled to a signal-generating particle 16.

The binding reagent 14 may be any reagent capable of specifically binding the desired analyte (e.g., lipopolysaccharide) and being mobilized from the conjugate region 12 during assay operation (i.e., to migrate along the test strip in the direction from conjugate region 12 to analytical membrane 18). In this first embodiment, the binding reagent 14 is an antibody, and more specifically, is a monoclonal antibody.

The signal-generating particle 16 that is coupled to the binding reagent 14 can be chosen from a plurality of materials (and may be chosen based on a desired mode of detection of the signal-generating particle 16). Examples of such particles include metallic particles, dye particles, fluorescent particles, and magnetic particles. In the first embodiment, the signal-generating particle 16 is a nanoparticle and, more specifically, may be a gold nanoparticle.

As described above, the binding reagent 14 with coupled signal-generating particle 16 is present on and/or in the conjugate region 12 (it may be present on and/or in the conjugate region 12 as manufactured, or it may be added during use). As liquid sample is added to the sample receiving region 30, it will migrate to the conjugate pad. If target analyte is present in the sample, it will be bound by the binding reagent 14 with coupled signal-generating particle 16. In certain embodiments, the binding reagent 14 with coupled signal-generating particle 16 may be part of a dried conjugate formulation. The conjugate region 12 comprises a porous conjugate pad containing the dried conjugate formulation. Upon contact with the liquid sample, the dried conjugate formulation is hydrated, and the binding reagent 14 coupled to the signal-generating particle 16 becomes mobile within the sample fluid.

The analytical membrane 18, in this first embodiment, is in fluid communication with, and disposed downstream of, the conjugate region 12. In some embodiments, the analytical membrane 18 comprises nitrocellulose. In certain embodiments, the analytical membrane 18 comprises a porous nitrocellulose membrane. The analytical membrane 18, as described above, includes both a test region 20 and a control region 22.

The test region 20 includes an immobilized capture reagent 24 configured to capture a complex 26 that includes the analyte (e.g., lipopolysaccharide) and the binding reagent 14. In this first embodiment, the immobilized capture reagent 24 comprises a polyclonal antibody. When target analyte is present in the sample, the binding reagent 14 coupled to the signal-generating particle 16 binds the analyte, forming a complex 26. This complex 26 migrates along the analytical membrane 18 to the test region 20 and is captured there by the immobilized capture reagent 24, resulting in accumulation of the signal-generating particle 16 at the test region 20 and formation of a detectable signal. In various embodiments, the signal-generating particle 16 may be selected to provide an optically visible signal, optical measurable signal, or machine-readable signal. In some embodiments, the signal is used for qualitative detection of analyte (i.e., whether analyte is present or not present in the sample). In some embodiments, the signal is used for quantitative determination of analyte (i.e., a measurement of some characteristic of the analyte, e.g., amount, concentration, change in concentration, etc.).

And the control region 22 includes a control component 28 configured to capture the binding reagent 14 coupled to the signal-generating particle 16. In certain embodiments, the control region 22 is positioned downstream from the test region 20. The control component 28 is immobilized on and/or in the analytical membrane 18 at the control region 22. In a specific embodiment, the control component 28 comprises protein G immobilized in the control region 22, which captures the binding reagent 14 coupled to the signal-generating particle 16, as the binding reagent 14 migrates along the analytical membrane 18. This results in accumulation of the signal-generating particle 16 at the control region 22 and formation of a detectable signal. The detectable signal can indicate proper sample migration, proper mobilization of conjugate, and general operability of the device 10.

Embodiments of the device 10 may also include an absorbent region 32. The absorbent region 32 is in fluid communication with the analytical membrane 18 and is configured to receive fluid that is migrating through the device 10. In that regard, the absorbent region 32 may thus serve to sustain capillary flow through the lateral flow assay device 10. Materials for such absorbent regions are generally known to those skilled in the art, and may comprise cellulose (or other suitable absorbent material).

In particular embodiments described herein, the sample includes saliva, and in more specific embodiments, may include human saliva. Further, in particular embodiments, the analyte to be detected and/or quantified is a lipopolysaccharide. Detection of lipopolysaccharide may be used to determine the presence and/or amount of bacterial endotoxin in the sample. I specific embodiments, the analyte may be lipopolysaccharide of P. gingivalis.

A second embodiment of this aspect of the invention is a lateral flow assay device for detecting the presence of, or quantifying an amount or concentration of, lipopolysaccharide in a sample. The device 10′ of this second embodiment (as shown in FIG. 1C) includes (1) a conjugate region 12′ including a binding reagent 14′ coupled to a signal-generating particle 16′, wherein the binding reagent 14′ comprises a first polyclonal antibody configured to bind lipopolysaccharide, and wherein the binding reagent 14′ is mobile relative to the device 10′; and (2) an analytical membrane 18′ in fluid communication with the conjugate region 12′. The analytical membrane 18′ includes a test region 20′ and a control region 22′. The test region 20′ includes an immobilized capture reagent 24′ configured to capture a complex 26′ including the lipopolysaccharide and the binding reagent 14′, wherein the immobilized capture reagent 24′ comprises a second polyclonal antibody. The control region 22′ comprises a control component 28′ configured to capture the binding reagent 14′ coupled to the signal-generating particle 16′. The device 10′ may also include a sample receiving region 30′ in fluid communication with the conjugate region 12′, and an absorbent region 32′ in fluid communication with the analytical membrane 18′.

The materials, arrangement, etc. of the components of this second embodiment may be the same as described above with respect to the first embodiment, with one general difference: While the first embodiment is directed to an embodiment where the binding reagent comprises a monoclonal antibody and the immobilized capture reagent comprises a polyclonal antibody—in this second embodiment, the binding reagent comprises a polyclonal antibody and the immobilized capture reagent comprises a polyclonal antibody.

Further, as described above in the Background section, the introduction of surface-enhanced Raman scattering (SERS) in lateral flow assays (LFA) has been recently exploited to increase the sensitivity and quantification capabilities of these rapid tests. However, there are still significant drawbacks to SERS-LFAs currently being used and researched, such as in the particular nanomaterials being currently used in such LFAs. Herein, the present inventors took advantage of a SERS-LFA combination to improve LFAs designed to test for P. gingivalis-a biomarker for oral health. As noted above, endotoxin released from a Gram-negative pathogenic P. gingivalis is causing chronic inflammation in the gum tissue and leading to tooth loss if not treated in time. In addition, some important cardiovascular and neurodegenerative diseases recently revealed a very close relationship to P. gingivalis concentration in oral cavity [63, 64]. Therefore, it becomes more important to monitor the PG LPS level for oral health as well as whole body health using a POC monitoring capability.

Previous work has reported the development of P. gingivalis colorimetric LFA detecting lipopolysaccharides (LPS) produced by the bacteria with a limit of detection (LOD) of ~22 ng/ml and a dynamic range up to ~10-20 μg/mL [31]. To utilize the LFA as a diagnostic test for early onset of oral diseases it is important to reduce the LOD below 10 ng/ml. Clinically, the average LPS concentrations of healthy individuals and periodontitis patients are approximately 10 ng/ml and 31 ng/ml, respectively [65].

Here the present inventors report on the combination of this LFA for P. gingivalis with a SERS readout with the LOD <10 ng/mL, which is within the range of concentration needed to monitor oral health. By comparison, conventional colorimetric LFAs achieve an LOD? 100 ng/ml, generating a lower diagnostic sensitivity. To achieve this enhanced sensitivity, the present inventors optimized the materials used in the SERS-LFA, investigating nanostars (NS) differing in size and material composition and comparing them to commercial gold nanoparticles, as a benchmark. More specifically, the present inventors explored five different metallic nanomaterials to determine the optimal nanomaterial for incorporation into SERS-LFAs: gold spheres/nanoparticles (AuNP), two sizes each of gold nanostars (AuNS) and silver-coated gold nanostars (Ag@AuNS). The Ag@AuNS has been shown to have superior single-particle signal enhancement although this property has not been tested in LFA systems yet. In particular, present inventors found that large (56-nm) NSs with a silver coating were the most sensitive nanomaterials for SERS-LFA.

To prove the applicability of this SERS-LFA to point-of-care (POC) settings, the present inventors tested the optimized LFA with a portable Raman system prototype designed to work on LFAs with 3D-printed cartridges and a line-shaped laser illumination. This work represents the first analytical comparison of more than two materials for optimization of SERS-LFAs. The different nanomaterials were first characterized and functionalized with a Raman reporter and the SERS enhancement of each nanomaterial was evaluated when collected at an LFA control line formed without the target PG LPS. Then, using the optimized nanomaterial, the SERS-LFA was calibrated for the detection of LPS produced by P. gingivalis and the sensitivity was compared to that of a colorimetric LFA. Using this prototype, the present inventors achieved the same LOD observed with the traditional benchtop Raman system. Thus, the portable Raman system described herein brings the SERS-LFA technology into the arena of POC use.

And so, the devices of the first and second embodiments described above, may also include components to allow those embodiments to operate as Raman-based lateral flow assays. To that end, in such embodiments, the signal-generating particle of the device of those embodiments is a SERS-active particle and is associated with a Raman reporter. As a result, once the binding reagent bound to analyte is captured at the test regions, the device is then configured to generate a Raman-detectable signal at the test region. In these embodiments, the SERS-active particle can be chosen from a gold nanoparticle, a gold nanostar, a silver-coated gold nanostar, and a combination thereof. And, in one particular embodiment, the SERS-active particle comprises a silver-coated gold nanostar. In one such embodiment, the silver-coated gold nanostar has an average size of about 56 nm. Further, due to the design and components used in such embodiments, the device is configured for Raman spectroscopic detection of analyte (e.g., lipopolysaccharide) at a concentration below about 10 ng/mL.

A particular embodiment of a device of this aspect (i.e., including SERS-LFA technology) includes (1) a blocking pad comprising a binding reagent coupled to a SERS-active particle and associated with a Raman reporter, wherein the binding reagent is configured to bind lipopolysaccharide, and wherein the SERS-active particle comprises a silver-coated gold nanostar; and (2) an analytical membrane in fluid communication with the conjugate region. The analytical membrane includes a test region and a control region. The test region includes an immobilized capture reagent configured to capture a complex comprising the lipopolysaccharide and the binding reagent. And the control region comprises a control component configured to capture the binding reagent coupled to the SERS-active particle. The device is configured to generate a Raman-detectable signal at the test region.

In this embodiment, the blocking pad may comprise fiberglass. Further, one or more blocking agents may be disposed on and/or in the blocking pad. The blocking agents may block the analytical membrane.

In this embodiment, the binding reagent comprises an antibody, and the antibody can be selected from a monoclonal antibody and a polyclonal antibody. Further, in this embodiment, the immobilized capture reagent comprises a polyclonal antibody.

Thus, in one specific embodiment of this SERS-LFA technology, the binding reagent comprises a monoclonal antibody and the immobilized capture reagent comprises a polyclonal antibody (with the monoclonal antibody of the binding agent conjugated to the SERS-active particle). And in another specific embodiment, the binding reagent comprises a polyclonal antibody and the immobilized capture reagent comprises a polyclonal antibody (with the polyclonal antibody of the binding agent conjugated to the SERS-active particle).

In a particular embodiment, the silver-coated gold nanostar has an average size of about 56 nm. Further, the device is configured for Raman spectroscopic detection of lipopolysaccharide at a concentration below about 10 ng/mL.

With the exception of the particular silver-coated gold nanostar described above, and the use of a blocking pad, as opposed to a conjugate region, the materials, arrangement, etc. of the components of this embodiment may be the same as described above with respect to the first embodiment.

A further aspect may include a combination including (1) the SERS-LFA device embodiment described above, and (2) a Raman scanner. In certain embodiments of this combination, the Raman scanner is configured to measure a SERS signal from the SERS-active particle along the lateral flow direction of the device with a selected resolution. The SERS signal may be generated via laser excitation. And, the laser may be focused on the test region and/or control region of the device. Further still, the excitation position of the laser can be adjusted to optimize the signal from the test region and/or control region. These and other principles of this embodiment will be explained in greater detail in the Examples, below.

Another aspect of the present invention provides a method of determining the presence of, or quantifying an amount or concentration of, an analyte (such as lipopolysaccharide) in a sample. Such a method may include first applying a sample to a lateral flow assay device (such as one of the embodiments of a device described above). For example, such a device may include a sample receiving region, a conjugate region, an analytical membrane having a test region and a control region, and an absorbent region, wherein the conjugate region comprises a binding reagent coupled to a signal-generating particle and configured to bind analyte, the test region comprises an immobilized capture reagent, and the control region comprises a control component (such as immobilized protein G). Next, the sample migrates through the device, where it forms (when analyte is present) a complex comprising analyte and the binding reagent coupled to the signal-generating particle. Such formation of complex may occur (or at least begin to occur at the conjugate region where the freely mobile binding agent/signal-generating particle is originally located.

As the sample (with analyte now bound to binding agent proceeds to migrate through the device, it will encounter the immobilized capture reagent an the test region of the analytical membrane. One may then detect a signal at the test region—that signal being provided by the signal-generating particle coupled to the binding agent. In some embodiments, detecting the signal comprises visually detecting a test line. In some embodiments, detecting the signal comprises optically measuring the test region. In some embodiments, detecting the signal comprises Raman spectroscopic detection.

The method then further comprises capturing additional binding reagent coupled to the signal-generating particle at the control region via use of immobilized control component at the control region.

In another aspect, the disclosure provides a method of quantifying analyte (such as lipopolysaccharide) in a sample. In such embodiments, the method comprises measuring a signal associated with the test region once the sample has migrated through the analytical membrane and test region such that any complex of analyte and binding reagent with signal-generating particle has bound to capture reagent in the text region. As another example, the mobile specific binding reagent is coupled to a SERS-active particle and associated with a Raman reporter, and detection or quantification is performed by measuring a Raman signal associated with the test region. One may then correlate the measured signal with a quantity of analyte in the sample. (In certain embodiments, such correlation may include comparing the measured signal to a reference value or calibration curve.)

Further aspects, explanation, and embodiments of the invention may be taken from the Examples, below.

### EXAMPLES

### Example 1

In this Example 1, a point-of-care/use lateral flow assay (LFA) is reported for the detection of P. gingivalis endotoxin, a major saliva biomarker for oral health. Two different approaches of sandwich LFA design using either the combination of mono- and polyclonal antibodies or polyclonal antibody only have been evaluated to detect P. gingivalis endotoxins, having a limit of detection of 22 ng mL-1 and 46.5 ng mL⁻¹ for water- and saliva-based samples, respectively. The LFA also exhibits good selectivity to P. gingivalis endotoxin versus other endotoxins and proteins. Saliva pretreatment combining syringe filtration and potato starch successfully inhibits α-amylase activity and provides improved results on LFA devices.

### Materials and Methods

### Materials

The standard gold nanoparticle solution (10 OD, 40 nm diameter) was purchased from Cytodiagnostics (Burlington, ON). Anti-P. gingivalis LPS monoclonal antibody produced in mouse, anti-P. gingivalis polyclonal antibody produced in rabbit, cellulose fiber sample/wicking pad (CFSP001700), Tween-20, Triton™ X-100, bovine serum albumin (lyophilized powder, ≥96%), amylase assay kits were purchased from MilliporeSigma (St. Louis, MO). Ultrapure LPS from P. gingivalis and E. coli were purchased from InvivoGen (San Diego, CA). Tris-HCl 1 M buffer solutions in different pH (7.0, 7.5, 8.0), sodium phosphate tribasic dodecahydrate (Na₃—PO₄·12H₂O, 98%, analysis grade), sucrose (certified ACS grade), sodium chloride (certified ACS grade) were all purchased from Fisher Scientific (Waltham, MA). Glass fiber pad (8950) for the conjugate membrane was purchased from Ahlstrom (Helsinki, Finland) and the nitrocellulose membranes (UniSart CN140 and CN95) were kindly provided by Sartorius (Goettingen, Germany). The backing card with dimensions of 60 (W)×300 (L) mm and 0.01″ thickness was purchased from DCN Dx (Carlsbad, CA). Human saliva was collected from healthy volunteers using the Pure SAL saliva collection kit obtained from Oasis Diagnostics (Vancouver, WA). Collected saliva was stored at −20° C.

### Conjugation of Antibody on Gold Nanoparticles

The passive adsorption method was used to conjugate gold nanoparticles to antibodies. This binding is “non-specific” and is governed by ionic, van der Waals, and hydrophobic forces. The optical density (OD) of concentrated 40 nm AuNP solution was evaluated using the NanoDrop One microvolume UV-vis spectrometer (Thermo Fisher Scientific) and adjusted to 10 OD. The solution of 200 μL of AuNP (10 OD) was mixed with 195 μL of 2.5 mM Tris-HCl pH 8 buffer with Tween-20 0.05% solution. Then, 5 μL of PG LPS antibody (1 mg mL⁻¹) was added to the solution. The mixed solution was placed in a 4° C. refrigerator and incubated overnight. To remove unbound free antibodies after incubation, the incubated solution was centrifuged at 1300 G for 20 min to form a pellet of Ab-AuNP conjugation at the end of the microtube and the supernatant of unbound antibodies was discarded. Centrifuged Ab-AuNP pellets were redispersed in fresh 2.5 mM Tris-HCl pH 8 buffer solution with 0.05% of Tween-20. This washing step was repeated 4 times. In the 4th washing step, centrifuged Ab-AuNPs were redispersed in 2.5 mM Tris-HCl pH 8 buffer solution with 0.05% of Tween-20 and 0.5% of BSA. The final optical density of conjugated Ab-AuNP solution was adjusted to 5 OD. The prepared solution was stored at 4° C.

### Fabrication of Lateral Flow Assay Devices

As illustrated in FIGS. 1A-1C, the LFA device consists of sample pad (cellulose fiber), blocking/conjugation pad (glass fiber), nitrocellulose membrane, and wicking pad (cellulose fiber). Each component was individually prepared before assembling on the adhesive backing card. All pads have the same width of 5 mm, while the length of sample pad, blocking pad, nitrocellulose membrane, and wicking pad are 13, 10, 30, and 17 mm, respectively.

Both sample and blocking/conjugation pads are pre-cut into the desired dimensions indicated above and then treated with multiple components before assembling on the backing card. Sample buffer solution for the sample pad was prepared by dissolving 7.5 μL of NaCl 1 M solution, 125 L of Triton X-100, and 2.5 mL of Tris-HCl 1 M buffer into 50 mL of ultrapure water. Cellulose pads were soaked into the prepared sample buffer solution for 30 min and dried at 50° C. for 90 min. Similarly, the blocking buffer solution for the blocking/conjugation pad was prepared by sequentially dissolving 250 mg of BSA, 500 mg of sucrose, 38.1 mg of Na₃PO₄, and 12.5 μL of Tween-20 surfactant into 5 mL of ultrapure water. Precut glass fiber pads were fully soaked in blocking buffer solution for 30 min then dried at 50° C. for 90 min.

For loading the Ab-AuNP conjugates on the conjugation pad, 18 μL of Ab-AuNP conjugated solution is dispensed on the conjugation pad loaded with blocking agents, and then dried again in the oven at 50° C. for 90 min.

A sheet of NC membrane (30 mm by 300 mm) and a strip of wicking (17 mm by 300 mm) pad were attached to the adhesive backing card. On the NC membrane, PG LPS antibody for the test line and protein G for the control line were printed using the Biodot AD1500 contactless aspirate dispense printing system (Biodot, Irvine, CA). Different concentrations of antibody were utilized for optimization and sample evaluations, while the concentration of protein G was fixed at 1 mg mL⁻¹ for most cases. After printing both materials on the NC membrane, the membranes are dried in an oven at 50° C. for 10 min and stored in a nitrogen-purged desiccator box overnight. The sheet of prepared cards printed with antibody and protein G is cut into thin strips 5 mm in width using Biodot CM⁴⁰⁰⁰ automatic guillotine cutter (Biodot, Irvine, CA). Finally, the treated sample and conjugation pads were assembled on the cut strips. Prepared LFA strips were stored in a nitrogen purged desiccator.

### Lateral Flow Assay Testing Process

Various concentrations of PG LPS were prepared in nuclease-free ultrapure water. For the structure in FIG. 1B, the sample solution was prepared by mixing 30 μL of mAb-AuNP conjugated solution, 10 μL of 3 M NaCl, 10 μL of PG LPS solution and 40 μL of Tris-HCl buffer solution with 0.05% of Tween-20. The LFA strips were placed on a light panel to obtain a consistent light environment. After 40 min incubation at room temperature, the sample solution was dispensed on the sample pad of the LFA strip. Images of both test and control lines were taken every 30 s for 30 min using a DSLR camera fixed on the tripod. When saliva samples were used, 40 μL of saliva replaces 40 μL of Tris-HCl buffer solution. For the second LFA structure in FIG. 1C, the sample solution was prepared by mixing 20 μL of 3 M NaCl, 10 μL of PG LPS solution and 60 μL of Tris-HCl buffer solution with 0.05% of Tween-20. For the saliva samples, 10 μL of saliva replaces 10 μL of Tris-HCl buffer solution.

### Pretreatment of Saliva Samples

Saliva was collected non-invasively from healthy volunteers using Pure SAL collectors, which removes a high percentage of mucinous materials, cell debris, food particles, etc. The collected saliva was subsequently treated through incubation with potato starch and/or filtration with/without potato starch. For incubation, the collected saliva was vortexed (2000 rpm) with the potato starch in 2:1 weight ratio and incubated at room temperature for the specific time period. After incubation, the mixture was centrifuged at 1.5 kG for 10 min and the supernatant was collected for LFA tests. For syringe filtration, the present inventors have used cellulose-based filter membranes with pore size of 0.2 or 0.45 μm. When necessary, potato starch was added to the syringe first, then the saliva was added to the potato starch-loaded syringe. LPS was spiked before the filtration process and the weight ratio of saliva and potato starch was fixed at 2:1 for all cases.

### Amylase Assay

Saliva was initially diluted 2000× with amylase buffer solution. 10 μL of diluted saliva was dispensed into wells of a 96 well plate followed by addition of 40 μL of amylase assay buffer to each well. Then, 100 μL of the Master Reaction Mix (amylase assay buffer: amylase substrate mix=50:50) is added in each well. The final solution volume in each well is 150 μL. After 2-3 minutes, the 96-well plate is placed into the reader at 25° C. The absorbance at 405 nm was recorded every 5 min until the absorbance of the most active sample becomes higher than that of the highest standard (20 nmole/well) for obtaining the linear relationship of results.

### Results and Discussion

### LFA Optimizations

Conjugation between antibody and AuNP can be readily accomplished by passive absorption, as described above. While AuNPs are quickly precipitated with polyclonal antibodies even at very low antibody concentrations, monoclonal antibodies formed a stable conjugation with AuNP even at NaCl concentrations as high as 500 mM. Tween-20 (0.05%) surfactant was added to prevent the absorption of Ab-AuNP conjugates on the microtube wall and aggregation of conjugates in solutions. Additionally, the use of buffer solution with proper pH and ionic concentrations provides significant positive effects on the stability of AuNP conjugates. When the present inventors used 2 mM Tris-HCl pH 8.0 buffer solution, very stable conjugates were formed even with a polyclonal antibody. Higher pH is beneficial for stable dispersion of AuNPs because it provides increased negative ion concentration surrounding gold surfaces, which results in stronger repulsion forces between particles [67]. Interestingly, when LPS is added to the stable pAb-AuNP conjugate solution, the conjugates are gradually aggregated over time, possibly because multiple pAb-AuNPs can bind to the single LPS.

For the first LFA approach (FIG. 1B), the present inventors utilized mAb for AuNP conjugation and pAb for the LFA test strip. Monoclonal antibody conjugated on AuNPs can provide good selectivity to PG LPS, while the polyclonal antibody acts as an effective capture probe for LPS bound to AuNP conjugates on the LFA device. In contrast to the case of pAb-printed LFA device, mAb printed on the LFA does not yield a test line because the mAb-specific binding site of LPS is already occupied by the same mAb present on the conjugated mAb-AuNPs.

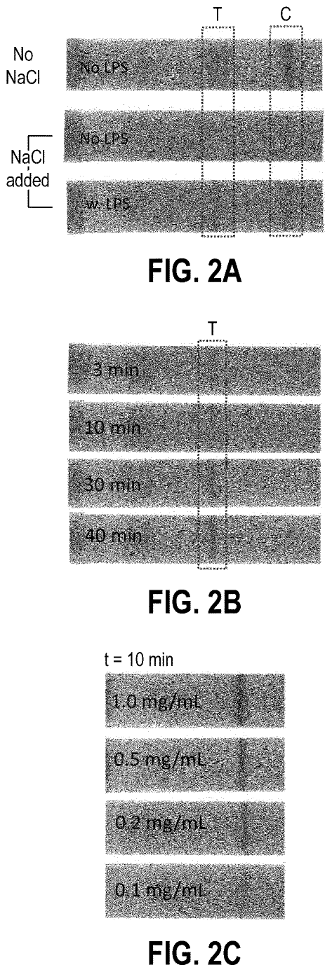

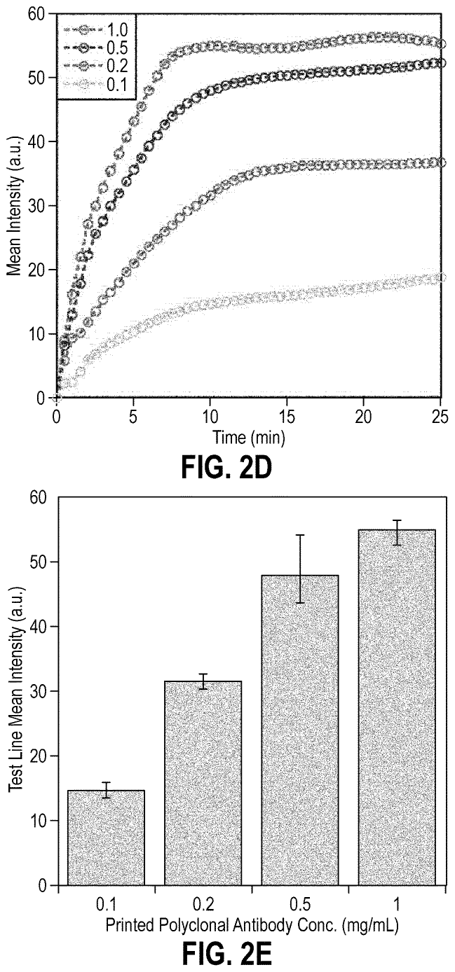

Additional optimization procedures were evaluated for enhancing the sensitivity of the assay. A simple mixture of mAb-AuNP conjugates and sample solution without LPS forms the false-positive test line (near the edges of the printed pAb area) (FIG. 2A, top strip). To remove the nonspecific binding effectively a combination of concentrated 333 mM NaCl solution, 0.05% of Tween-20, and 0.5% of BSA was utilized. Highly stable mAb-AuNP conjugates are required to avoid aggregation in the presence of high NaCl concentration. Because the mAb-AuNP conjugates that the present inventors have synthesized have an excellent stability at 333 mM NaCl concentration, non-specific binding (in absence of LPS) was effectively prevented (FIG. 2A, middle strip) while in the presence of LPS a test line was formed (FIG. 2A, bottom strip). Importantly, the mixtures of mAb-AuNP and sample solutions need to be incubated over a period long enough to provide sufficient time for the mAb-AuNP conjugate to capture LPS molecules. As shown in FIG. 2B, longer incubation times of 30-40 min led to noticeable increases in test line intensity. Therefore, although the test line intensities are similar between 30 and 40 min incubation time, the present inventors have incubated all mixtures for 40 min in all subsequent experiments to eliminate any inconsistency of test line intensity caused by potential small variations of the incubation time. The conditions for printing of the polyclonal antibody (pAb) on LFA were also optimized. For the pAb printing, the Biodot automated contactless bioprinter was utilized as described in Experimental section. The test line intensity increases with pAb concentration but begins to saturate at 1.0 mg mL⁻¹, as shown in FIG. 2C. Test line intensity increases rapidly with time in the first few minutes after sample dispensing (FIG. 2D) and reaches a steady state level in 10-15 min. The pAb concentration of 1.0 mg mL⁻¹ was selected to maximize the test line intensity and to minimize the possible variation from batch to batch of printed pAb (FIG. 2E).

### LFA Evaluation for LPS in Aqueous Sample Solutions

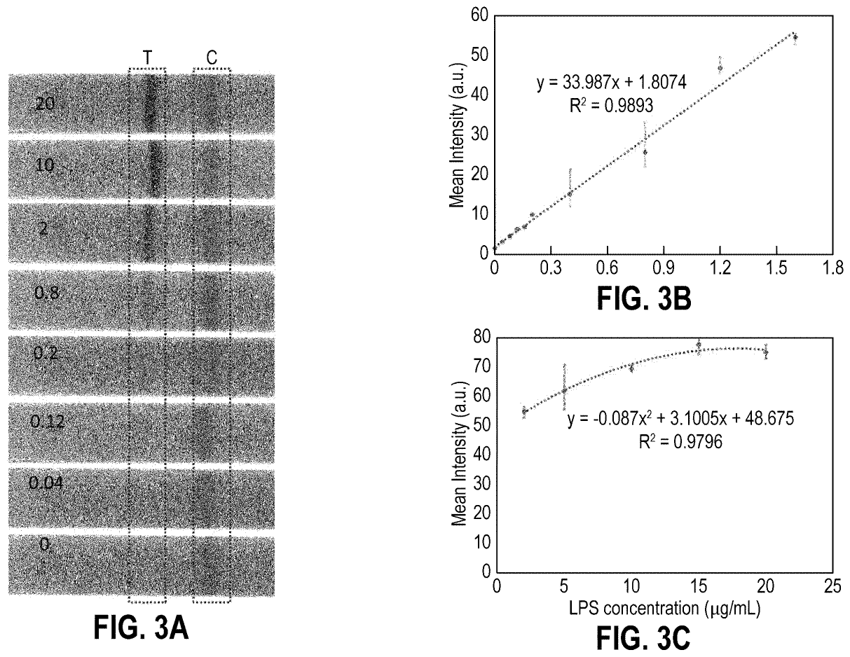

After the optimization of the antibody conjugation and immobilization was completed, the sensitivity of the LFA assay with different LPS concentrations was evaluated. Sample solutions dispensed onto LFA devices consisted of 30 μL of mAb-AuNP conjugation, 10 μL of sodium chloride 3 M concentration, 10 μL of PG LPS solution (of varying concentrations from 0 to 20 μg mL⁻¹), and 40 μL of ultrapure water. As shown in FIG. 3A, the sandwich immunoassay approach forms clear test and control lines. The test line intensity increases monotonically with LPS concentration, while the control line intensity is approximately constant.

Quantitative results can be extracted using Image J software (National Institute of Health). The mean intensities of the test line area and adjacent empty area (background) were first measured. The final test line intensity was obtained by subtracting the mean background value from the mean value of the test line area.

FIGS. 3B and 3C show the quantified test line intensity at multiple LPS concentrations. The test line intensity increases linearly with LPS concentration in the low μg mL-1 range (FIG. 3B) and then saturates in the 10-20 μg mL⁻¹ concentration range (FIG. 3C). An effective linear dynamic range of 75×, up to 1.6 μg mL⁻¹ concentration is obtained. The limit of detection (LOD) of 22 ng mL⁻¹ is calculated. LOD is the lowest analyte concentration which can be distinguished from a limit of blank (LOB, no LPS case) [68].

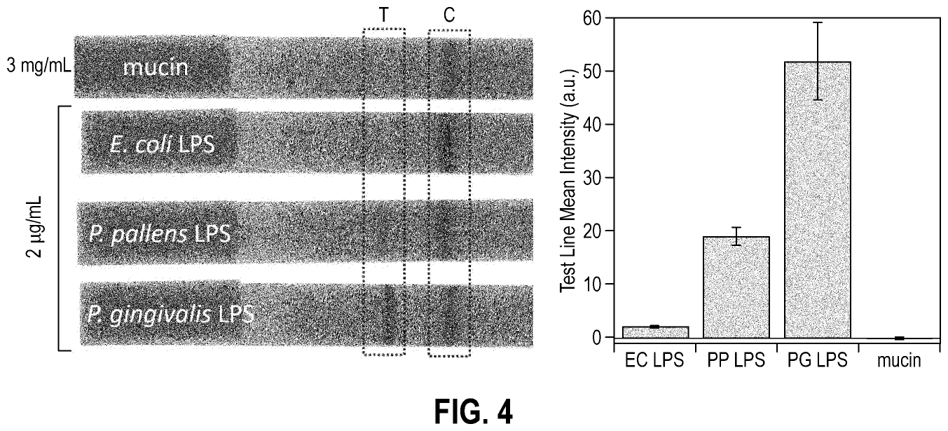

An initial evaluation of the selectivity of the assay was performed. As shown in FIG. 4, LFA presented the strongest test line intensity for P. gingivalis. The other LPS tested, P. pallens, resulted in a much weaker test line. However, that would not be an issue in the practical use for oral health evaluation because P. pallens LPS is also related to oral disease, especially for gingivitis. Mucin, which is the most abundant protein in saliva, and E. coli LPS did not produce a test line signal.

### Pretreatment of Saliva Samples for LFA Test

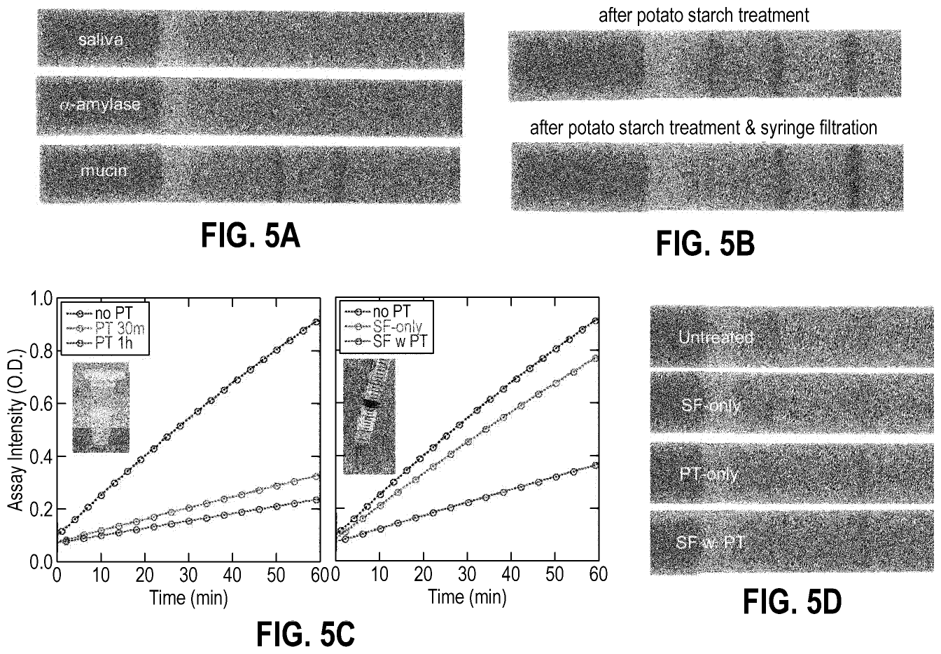

After the evaluation of the LFA devices with water-based samples spiked with LPS, the present inventors have used LPS-spiked saliva samples from healthy volunteers. Using untreated whole saliva spiked with PG LPS in the LFA device, test and control lines are not formed (FIG. 5A, top strip), most likely due to interference from other biomacromolecules present in saliva. Because a-amylase and mucin are the most abundant biomolecules in saliva, they were tested individually in LFAs, without using saliva (FIG. 5A, middle and bottom strips). Mucin and a-amylase were dissolved into water at concentrations found in saliva of 0.3% and 0.27%, respectively. Interestingly, mucin does not appear to have a negative effect on the P. gingivalis-antibody reactions in the LFA since the test and control lines are clearly visible and well-defined (FIG. 5A, bottom strip). On the other hand, adding a-amylase to the P. gingivalis LPS solution obstructed the immunoreactions, preventing the formation of both test and control lines, similarly to the saliva test result. Mitigating a-amylase from the saliva 32 is clearly a very important consideration for salivary endotoxin detection in antibody-based LFA tests.

Amicon ultrafiltration with 100 kDa filter effectively removes most biomolecules including α-amylase. However, because the P. gingivalis LPS exists as a large aggregation >100 kDa, Amicon ultrafiltration cannot be utilized to retain PG LPS while removing α-amylase from the saliva. Instead, other approaches were investigated. Syringe filtering of the saliva was performed using filters with 0.2 or 0.45 μm pore size. Interestingly, after syringe filtering, test and control lines are formed for both 0.2 and 0.45 μm pore sizes although the test line intensity is weaker than that of aqueous solution case.

It has been reported that α-amylase can be removed by using potato starch because of its high affinity to α-amylase [69]. Potato starch has been utilized as a substrate for the α-amylase inhibition assay. Saliva was mixed with potato starch in a 2:1 wt. ratio and incubated for 30 min. After incubation, the mixture was centrifuged at 1.5 kG for 10 min and the treated saliva was collected from the supernatant.

After potato starch treatment, test and control lines are formed on the LFA during tests with P. gingivalis in saliva, as shown in FIG. 5B (top). This indicates that the potato starch effectively removes α-amylase while it does not affect the LPS. However, some non-specific binding was observed near the test line, and the flow of the treated saliva on the LFA was not uniform possibly due to the presence of potato starch in saliva samples. To remove any potato starch component and undesired biomolecules in saliva, the treated saliva was filtered prior to dispensing on the LFA using cellulose-based syringe filters with 0.45 μm pore size. This combined treatment reduces the non-specific binding and noticeably improves the line intensities, as shown in FIG. 5B (bottom). To evaluate the effect of potato starch and syringe filtration on α-amylase level, the present inventors have utilized the amylase activity assay kit as described above. The determination of amylase activity utilizes a coupled enzymatic assay that generates a colorimetric output at 405 nm wavelength. The colorimetric signal is directly proportional to the quantity of cleaved substrate, ethylidene-pNP-G7, by the amylase. As shown in FIG. 5C, amylase level is significantly reduced to ~30% after 30 min incubation with potato starch. Longer incubation results in minor additional reduction in amylase level, but 30 min incubation seems a good operating condition. Syringe filtration by itself reduces the amylase level only slightly. However, adding potato starch to the syringe yields significant reduction in amylase level even without any time-consuming incubation. For incubation with potato starch, saliva samples were mixed with potato starch in 2:1 wt. ratio, then vortexed at 2000 rpm for desired time. The same amount of potato starch (200 mg) was added to the syringe to filter 400 μL of saliva sample. Clearly, the test line intensity after various pretreatments (FIG. 5D) is related to the level of amylase present.

### LFA Evaluation for LPS in Pre-treated Saliva Sample Solutions

Using the improvements obtained by saliva treatment described above, P. gingivalis LPS detection at different concentrations in saliva was tested on LFA devices. Saliva was first spiked with LPS and then treated with potato starch and syringe filtration. Results were obtained for LPS concentrations from 0.5 to 2 μg mL⁻¹ and for a control experiment without any LPS. The LFA assay is sensitive to the presence of LPS in saliva. The test line intensity variation with increasing LPS concentration can provide a quantitative measure of the LPS concentrations in saliva. However, overall test line intensity is still weaker than that of water-based samples (FIG. 3A).

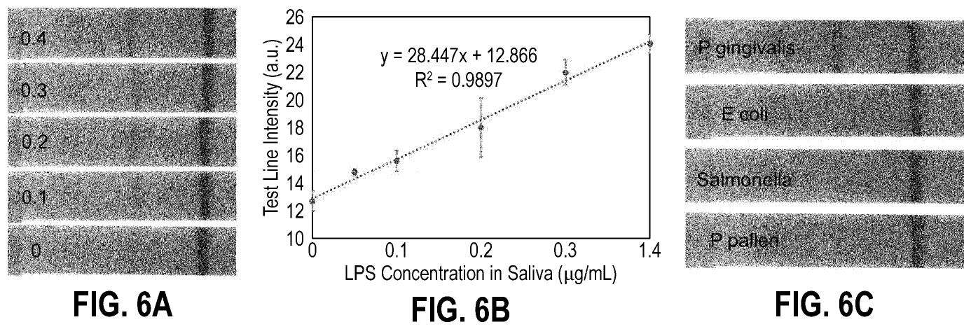

As mentioned above, the first sandwich LFA design is not very practical for POC application because of the long incubation time. Also, the sensitivity is limited by the currently available monoclonal antibody for PG LPS. To improve the sensitivity and address the incubation time issue, the second LFA design (FIG. 1C) was utilized for treated saliva samples as shown in FIGS. 6A-6C. Interestingly, all AuNP conjugates are effectively released from the conjugation pad, establishing strong test and control lines, as shown in FIG. 6A. In the first design, a significant amount of AuNP conjugates remains at the sample pad (reddish color, FIG. 5A-5D), while no reddish color remains in the conjugation pad of the 2nd design (FIG. 6A), which indicates most of AuNP conjugations were released through the nitrocellulose membrane. Quantitative analysis using ImageJ (FIG. 6B) presented the LOD of 46.5 ng mL⁻¹, which is comparable to that of water-based samples (22 ng mL⁻¹). The excellent selectivity among other bacterial LPS is obtained as shown in FIG. 6C.

### Summary and Conclusion

In this Example 1, the present inventors have demonstrated an antibody-based sandwich LFA assay to detect P. gingivalis LPS, a major biomarker for oral health. The LFA device has LOD of 22 ng mL⁻¹ for aqueous samples and excellent selectivity versus other LPS and salivary proteins. Detection in human saliva was evaluated in combination with potato starch and syringe filtering to reduce the interference from biomolecules in saliva, especially for α-amylase enzymes. LFA immunoassay reactions with saliva samples resulted in comparable results (LOD 46.5 ng mL⁻¹) to that of water-based samples.

Further the pretreatment for saliva samples discussed above can be also utilized with aptamer-based sandwich LFA devices.

### Example 2

As described above, the introduction of surface-enhanced Raman scattering (SERS) in lateral flow assays (LFA) has been recently exploited to increase the sensitivity and quantification capabilities of these rapid tests. Herein, the present inventors took advantage of a SERS-LFA combination to improve LFAs designed to test for P. gingivalis, a biomarker for oral health. The present inventors have demonstrated a limit of detection (LOD)<10 ng/mL, which is within the range of concentration needed to monitor oral health. By comparison, conventional colorimetric LFAs achieve an LOD≈100 ng/mL, generating a lower diagnostic sensitivity. To achieve this enhanced sensitivity, the present inventors optimized the materials used in the SERS-LFA, investigating nanostars (NS) differing in size and material composition and comparing them to commercial gold nanoparticles, as a benchmark. The present inventors found that large (56-nm) NSs with a silver coating were the most sensitive nanomaterials for SERS-LFA. To prove the applicability of this SERS-LFA to point-of-care (POC) settings, the present inventors tested the optimized LFA with a portable Raman system prototype designed to work on LFAs with 3D-printed cartridges and a line-shaped laser illumination. Using this prototype, the present inventors achieved the same LOD observed with the traditional benchtop Raman system. The use of a portable Raman system has brought the SERS-LFA technology closer to the POC use.

### Materials

Hydrochloric acid (1 M), chloroauric acid solution (HAuCl₄, 5.08 mM), ammonium hydroxide (NH₄OH), 1,1′,3,3,3′,3′-hexamethylindotricarbocyanine iodide (HITC), Tween 20 (10% w/v), Triton™ X-100, L-ascorbic acid, anti-P. gingivalis LPS monoclonal antibody, anti-P. gingivalis polyclonal antibody, bovine serum albumin (BSA), and cellulose fiber pad (CFSP001700) were purchased from Sigma-Aldrich (St. Louis. MO). Silver nitrate (AgNO₃), sodium chloride, sodium phosphate tribasic dodecahydrate (Na₃PO₄·12H₂O, 98%, analysis grade), sucrose, Tris-HCl (1M, pH 8), Tris-HCl (1M, pH 7.4), phosphate buffered saline (PBS, 10×, pH 7.4), trisodium citrate dihydrate, and 4-mercaptobenzoic acid were purchased from Fisher Scientific (Waltham, MA). Thiolated polyethylene glycol (HS-PEG, MW≈5000 k) was purchased from Nanocs (New York, NY). The standard 40 nm gold nanoparticle solution (10 OD) was purchased from Cytodiagnostics (Burlington, ON). All ultrapure P. gingivalis and E. coli LPS were purchased from InvivoGen (San Diego, CA). Glass fiber pad (8950) and nitrocellulose membrane (CN140 and CN95) were obtained from Ahlstrom (Helsinki, Finland) and Sartorius (Goettingen, Germany), respectively. The backing card in a size of 60 (W)×300 (L) mm was purchased from DCN Dx (Carlsbad, CA). All materials were used as received and solutions were prepared using Millipore Synergy UV-R Ultrapure water (18.2 MΩ·cm).

### Preparation of Gold Nanostars and Silver-Coated Nanostars

Gold seeds were synthesized by the Turkevich method (approx. 12 nm) [70]. AuNS and Ag@AuNS were prepared according to previously published protocols [71]. For large nanostars, 2.6 μmoles of HAuCl₄ and 100 μL of Au seed solution were added to 10 mL of 1 mM HCl with vigorous stirring. To form nanostars, AgNO₃ (0.125 mmoles) and L-ascorbic acid (5 mmoles) were added to the reaction flask in rapid succession, resulting in a color change from pale orange to deep blue. To make Ag@AuNS, 5 mmoles AgNO₃ and 10 μL NH₄OH were added in rapid succession to the blue nanostar solution and the flask was stirred until the solution was red-brown and the color was stable. Particle solutions were stored at 4° C. until use.

To produce smaller NS, the same procedure was followed with the volume of Au seed solution increased to 200 μL. All other additions were constant.

Functionalization of Nanoparticles with Raman Reporter and Antibody

A 1 mL aliquot of the nanomaterial (i.e., AuNP, AuNS, or Ag@AuNS) was combined with 100 μL of 5 μM HITC and the mixture was incubated for 3 hours at RT. After incubation, Tween 20 was added to achieve a final surfactant concentration of 0.01% (v/v). The samples were well mixed and centrifuged at 4000 g for 10 min to separate the particles from excess Raman reporter in solution. The supernatant was removed and the particles were redispersed in 0.01×PBS buffer. After conjugating the Raman reporter, 5 μL of PG LPS monoclonal antibody (1 mg/mL) was added to 200 μL of nanomaterial solutions. Then 195 μL of 2 mM Tris-HCl pH 8.0 with 0.05% of tween-20 surfactant was also added. The mixed solutions were incubated overnight in the refrigerator. After incubation, the solutions were centrifuged at 1,300 g for 20 min, followed by the removal of the supernatant. This washing process was repeated 4 times. After the 4th washing step, the concentrations of nanomaterials were adjusted to 5 OD using the buffer made of 2-mM Tris-HCl pH 8.0, 0.05% of tween-20, and 0.5% of BSA.

### Preparation of LFA Strips

The present inventors' LFA strip has 4 components-cellulose sample pad, fiberglass blocking pad, nitrocellulose (NC) membrane, and cellulose wicking pad. No “conjugation” pad was present because AuNP/AuNS are not loaded on the pad. Blocking agents were loaded to the (blocking) pad to block the nitrocellulose membrane.

First, the NC membrane (30 mm by 300 mm) and wicking pad (17 mm by 300 mm) are fixed on the adhesive backing card, and then cut into 5-mm wide strips using the Biodot CM4000 automatic guillotine cutter (Biodot, Irvine, CA). Next, P. gingivalis polyclonal antibody for the test line and protein G for the control line are printed using the Biodot AD1500 printing system (Biodot, Irvine, CA). Both concentrations were optimized at 1 mg/mL. Printed strips were incubated in an oven at 50° C. for 10 min and stored in a nitrogen-purging box.

Both sample and blocking pad were precut into 13 mm×5 mm and 10 mm×5 mm, respectively, and loaded with multiple functional chemicals to provide essential functions (e.g., easy uniform flow, preventing non-specific binding, etc.) during LFA test. Sample pads were immersed into the buffer solution dissolved with 2.5 mL of Tris-HCl 1 M pH 8 buffer, 2.5 μL of NaCl 3 M solution, and 125 μL of Triton X-100 into 50 mL of ultrapure water. Blocking pads were immersed into blocking buffer solution prepared by sequentially dissolving 250 mg of BSA, 500 mg of sucrose, 38.1 mg of Na₃POa tribasic salt, and 12.5 μL of Tween-20 surfactant into 5 mL of ultrapure water. Both pads were immersed into the solution for 40 min, dried in the oven at 60° C. for 90 min, and stored in a nitrogen purging box overnight. To complete the fabrication, pretreated sample and blocking pads were placed on the backing card pre-assembled with the NC membrane and wicking pad before LFA tests.

### Nanoparticle Tracking Analysis

Nanoparticle tracking analysis was performed with a Nanosight NS300 (Malvern Panalytical, Worcestershire, UK) using Nanosight NTA software. Five replicate measurements of each sample were collected for 30 seconds each.

### Transmission Electron Microscopy

Before TEM imaging, 10 μL of the nanomaterial solution was dispensed onto a 300-mesh grid and dried overnight. A transmission electron microscope (TEM) Talos F200i (Thermofisher) at 200 kV was used to acquire the micrographs. This instrument was used at the University of Cincinnati's Advanced Material Characterization Center (AMCC).

### UV-Vis Spectroscopy

UV-vis absorbance spectra were collected in a 96 well plate with a Synergy HTX multi-mode plate reader (BioTek, Winooski, VT) and Gen5 software (v3.10). Measurements were collected from 300-800 nm and blank subtracted in the native software.

SERS Measurements with Benchtop Raman

SERS measurements were collected on a lab-built Raman setup composed of a Wasatch Photonics WP785 spectrometer, an OptoEngine 785-nm laser source (FC-D-785-300 mW) and Raman fiberoptic probe (RamanProbe by InPhotonics). Laser power was set to 74 mW for the measurements. The fiberoptic probe was connected to a 2D automated stage (Zaber). The stage was used to collect spectra on the LFA. Spectrum collection for material optimization was performed first as a single line-scan along the sample flow direction in the LFA (40 points 1 mm step-size) to identify the location of the control line. Then 3 parallel scans (of 10 points each) were performed along the control line to maximize SERS spectral information.

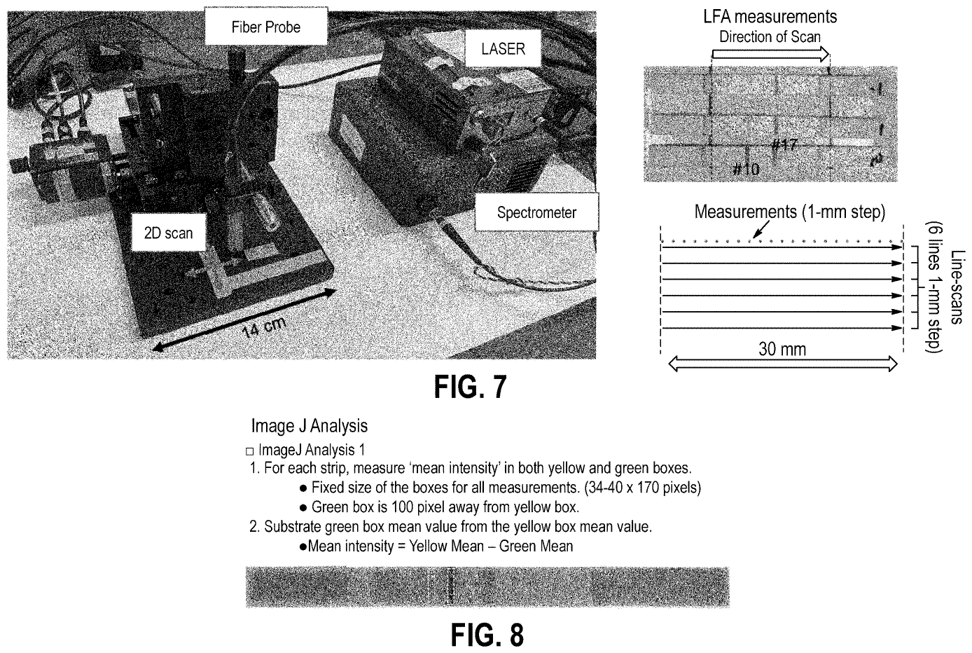

For the LFA operation with the targeted analyte, detecting spectra were acquired along the LFA in 6 parallel line-scans (40 points 1 mm step-size) separated by 1 mm. The line scans were averaged in the displayed results. Each measurement had 100 ms integration time for 10 accumulations. FIG. 7 shows a photo of the system and a depiction of the sampling on the LFA.

SERS Measurements with Portable Raman

To prove the POC capability of the present inventors' test, SERS measurements were also collected on a portable Raman system. The system is a WP-785XS prototype provided by Wasatch Photonics. This system is composed of a 785-nm laser of 8 mW in power, holographic grating and an ambient CMOS detector. This portable system has a size of roughly 55×85×30 mm. The excitation/collection optics are designed to focus on a 3.5-mm line (0.5 mm thick), which is ideal for reading SERS signal from LFA test lines. The system was integrated with custom 3D-printed cartridge and sample holder. The cartridge permitted to enclose the LFA and the sample holder kept the cartridge/LFA at the focal distance. Photos of system, cartridge and sample-holder are shown later in the results section. The laser spot for the benchtop Raman system (−1 mm) has a power density of 19 W/cm², compared to the line of the portable system with a power density of 4 W/cm². The focal distances for the two systems are 5 and 21 mm for the benchtop and portable, respectively.

### Image and Data Analysis

LFA colorimetric results were analyzed using ImageJ (V2.1.0/1.53c) [72]. FIG. 8 describes the image analysis process. Raman data were processed using scripts running in MatLab (R2021b) (The Mathworks, Inc., Natick, MA). A lab-built script with smoothing using a Savitzky-Golay frequency filter and partial least squares baseline subtraction was used prior to data analysis. The SERS response was determined from the normalized peak intensity for the characteristic HITC band at 546 cm⁻¹. Data visualization was executed in IGOR Pro (v8.0.4, Wavemetrics, Inc., Lake Oswego, OR).

### Results and Discussion

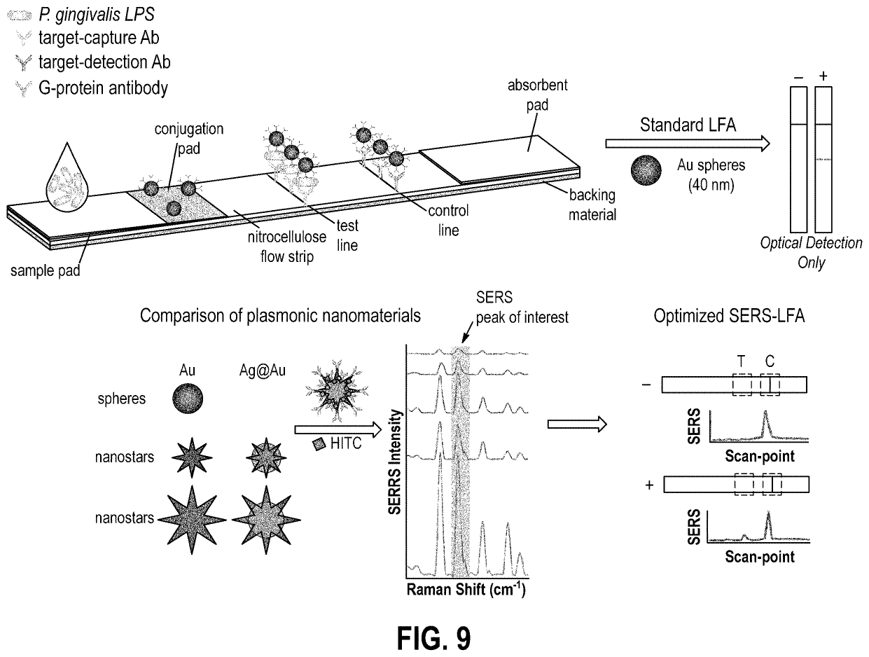

Conventional operation LFAs rely on visual detection of the test line generated by collected AuNP. To improve the performance of the LFA, SERS active reporter molecules can be incorporated on the metal surface. It has been reported that the surface plasmon of nanomaterials with higher anisotropy produces stronger SERS response than that of spherical AuNP [73]. However, this effect has not been carefully studied in LFA strips. Therefore, the present inventors have synthesized AuNS and Ag@AuNS in different sizes for comparison to commercially available AuNP. FIG. 9 shows a graphical depiction of the LFA operation and the optimization of SERS reporter conjugation on nanomaterials.

### Characterization of Plasmonic Nanomaterials

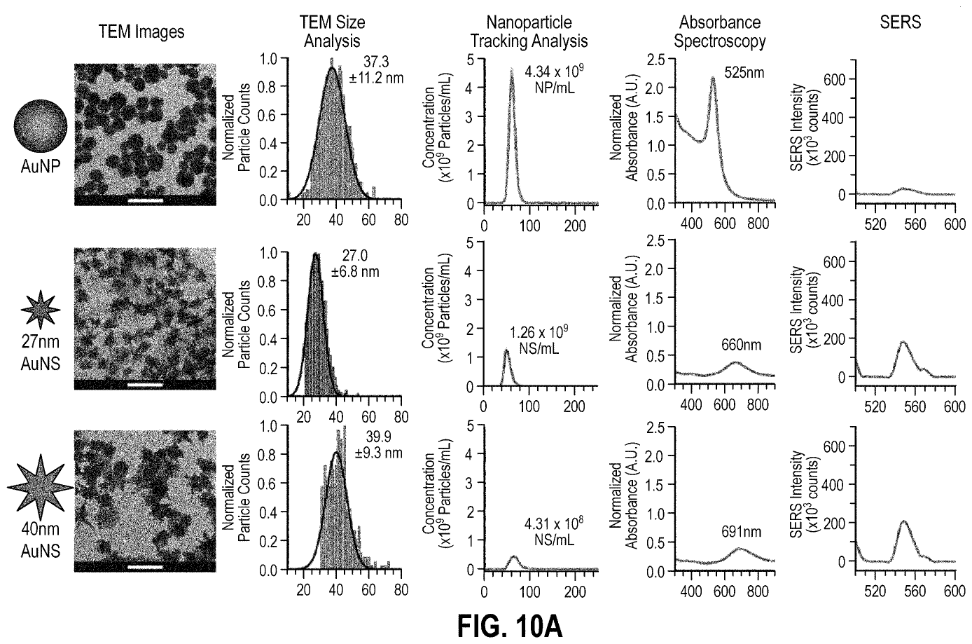

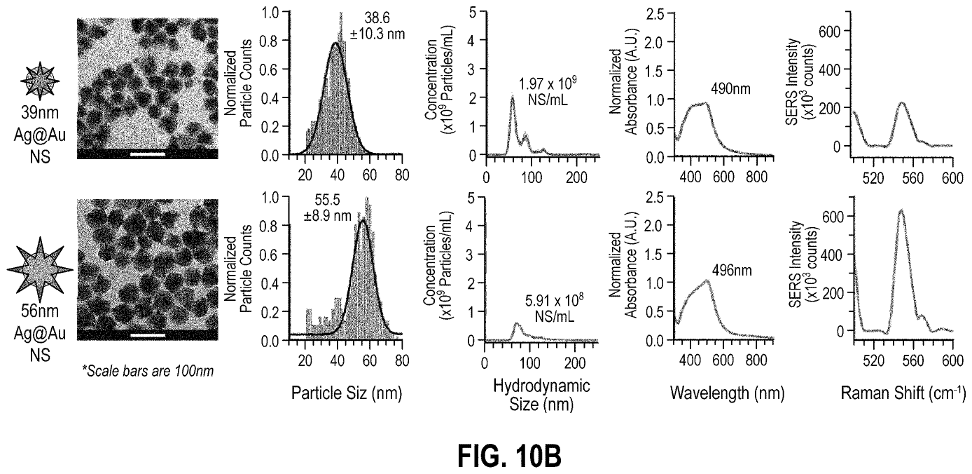

To understand the structural and plasmonic properties of the nanomaterials tested, each material was analyzed by UV-vis, NTA, and TEM. All characterization data are shown in FIG. 10 FIGS. 10A and 10B. From the TEM images, the morphology and size of each material was determined. The TEM images show that the commercial AuNP are spherical and the average diameter was 37.3±11.2 nm. The AuNS display the highly branched structure, with average particle diameters (tip-to-tip) of 27.0±6.8 and 39.9±9.3 nm for the small and large AuNS, respectively. Finally, the Ag@AuNS appears somewhat rounded, although contrast shows the branched Au structure is intact beneath the Ag shell; these particles had average diameters of 38.6±10.3 and 55.5±8.9 nm, for the small and large Ag@AuNS, respectively. Hereafter, these AuNS will be referred to as 27 and 40 nm and their Ag-coated analogs will be referred to as 39 or 56 nm. From the TEM size analysis, the size distributions of all lab-prepared NS were comparable to the commercial AuNP, indicating control over the synthesis and a product of particles with uniform size.

Nanoparticle tracking analysis (NTA) provides a rapid assessment of size and concentration of a given nanoparticle solution. Although the size assessment is rapid, it is based on hydrodynamic radius, resulting in measurements that differ from the results of TEM image analysis. Pure Au nanomaterials were stabilized by a surface layer of thiol-PEG, contributing an average of 6 nm to the size measurements. The hydrodynamic sizes of all particles were on average 18 nm larger than the measurements taken by TEM.

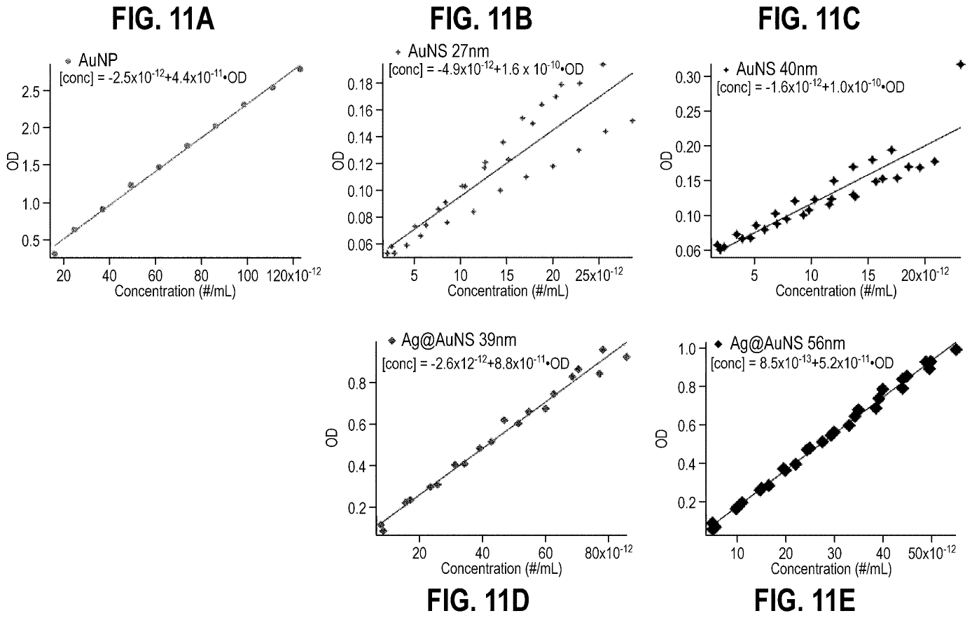

The NTA results in FIG. 10 FIGS. 10A and 10B show that the as-synthesized concentration of 27 nm AuNS and 39 nm Ag@AuNS is 30-45% that of commercially available AuNP; as-synthesized 40 nm AuNS and 56 nm Ag@AuNS are 10-13% of the commercial AuNP concentration (this lower concentration was expected due to the reduced number of nucleation sites provided via Au seeds). The concentration data provided by NTA is especially useful because it provides the ability to ensure the samples being compared are similar. NTA measurements are relatively fast, but not practical for measuring every sample, thus the present inventors measured the concentration of several batches of nanostars and calibrated the corresponding UV-vis absorbance intensities against them; with this information the present inventors are able to rapidly assess the concentration of each type of nanomaterial via absorbance and adjust concentrations as needed for comparable samples. The calibration of various nanomaterials is provided in FIG. 11A-11E. Calibration and absorbance data were used to keep the concentration of nanomaterials the same in the SERS comparison and for LFA loading.

The UV-vis absorbance spectrum of the nanomaterials also provides some indication of the plasmonic properties for the pure gold nanomaterials. Previous work has shown that the localized surface plasmon (LSP) of AuNS shifts over time, and that SERS performance correlates to the observed LSP from UV-vis absorbance measurements [74]. For AuNP, the absorbance maximum is located at 525 nm, corresponding to the LSP of the materials. Thus, the SERS enhancement for AuNP is optimal with laser excitation near 525 nm. The as-synthesized AuNS, however, showed LSP bands at 660 and 691 nm for the 27 nm and 40 nm AuNS, respectively. Thus, these materials are expected to perform optimally with laser excitation around 650 nm. When Ag-coating is added to the surface of the NS, the LSP band is shifted, as the Ag surface dominates the optical properties. In these cases, however, the present inventors anticipate the LSP to correspond to those of the uncoated materials, because (as shown in the TEM images) the Au core structure is intact.

The present inventors' first comparison of SERS performance between different nanomaterials was conducted by measuring the signal of reporter-functionalized particles on the cellulose membrane used for LFA sample pads. Although the LSP bands for all of the materials studies are centered between 500 and 600 nm, the Raman reporter selected, HITC, shows resonance response with a 785-nm laser. The SERS data in FIG. 10 FIGS. 10A and 10B shows the region-of-interest for the reporter for each nanomaterial and Table 1 provides a summary of the enhancement differences between nanomaterials. The average SERS intensity of the reporter on AuNP was 2.9×10³ counts. The signal intensities of 27 and 40 nm AuNS were ~6.2× and 6.9× higher than AuNP, respectively. For 39 and 56 nm Ag@AuNS, the signals were further enhanced to ~8.6× and ~21.4× higher than that of AuNP, respectively.

TABLE 1
SERS enhancement factors relative to AuNP.

| | On Conjugation Pad | | Control Line on LFA | |
|---|---|---|---|---|
| | Avg. SERS | | Avg. SERS | |
| | Intensity | EF relative | Intensity | EF relative |
| Nanomaterial | (St. Dev) | to AuNP | (St. Dev) | to AuNP |
| AuNP | 2865 | — | 1224 | — |
| | (164) | | (1921) | |
| 27 nm AuNS | 17656 | 6.16 | 3266 | 2.67 |
| | (1550) | | (3912) | |
| 40 nm AuNS | 19831 | 6.92 | 11224 | 9.17 |
| | (1951) | | (3769) | |
| 39 nm | 24575 | 8.58 | 15758 | 12.88 |
| Ag@AuNS | (2666) | | (3947) | |
| 56 nm | 61259 | 21.39 | 22807 | 18.64 |
| Ag@AuNS | (8764) | | (8268) | |

### Comparison of SERS in LFA

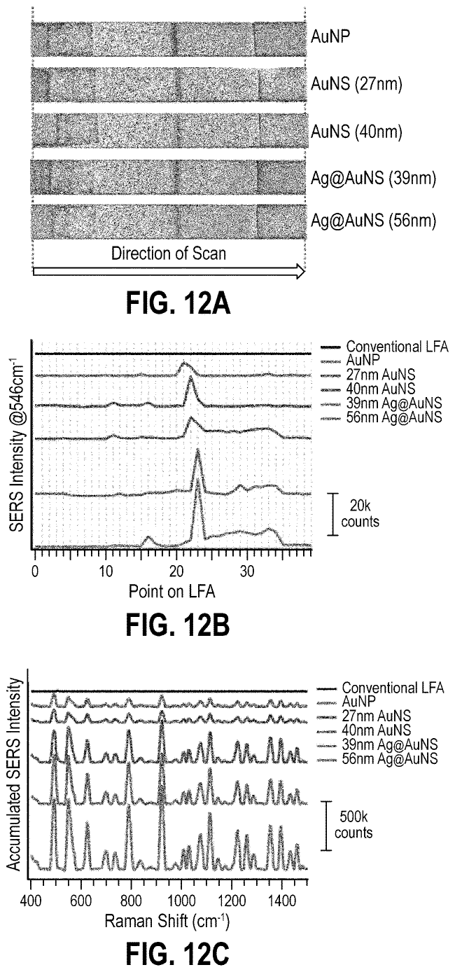

To investigate the performance of each nanomaterial, the present inventors prepared LFAs with only a control line and analyzed the SERS signal across the length of the LFA strip. Photographs of post-test LFAs are shown in FIG. 12A. The images show that the LFA with AuNP produced a stronger (darker) optical control line than the LFAs with NS. This may be an indication that spherical particles flow better through the test strips than the nanostars whose physical features may become entangled in the network of the NC membrane. However, the SERS improvement of NS over NP (FIG. 12B) suggested the signal at the test line would still be significantly better for NS test strips than for NP test strips.

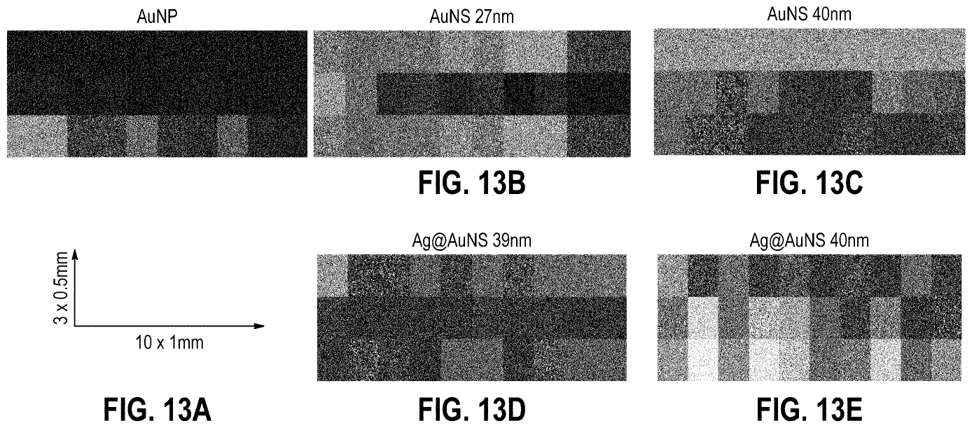

To analyze the SERS signal from the LFAs, the present inventors employed an automated Raman scanner, which was programmed to measure the SERS signal along the length of the LFA (sample flow direction) with a chosen resolution (in this case, 1 mm increments over the 40 mm total length). This line scan is indicated by the arrow in FIG. 12A. The scanner shows in real-time signal intensity at each point (FIG. 12B). The position of the control line was used to select the point location for collecting three parallel 10-point scans across the width of the LFA to provide accumulated SERS spectra for the control line of each test strip (shown in FIG. 12C as accumulated spectra). In total, completion of the measurements require ~5 min. FIG. 13A-13E displays the maps obtained from the single data points in these measurements, to show the spatial variability.

The results on the LFA test strips show that the general trend for SERS improvement holds true after flow through capillary action (Table 1). The accumulated signal on the control line from AuNP was −1200 counts. For the 27 and 40 nm AuNS, SERS signals were ~2.7 and 9.2× greater than that of the commercial AuNP, respectively. For the 39 and 56 nm Ag@AuNS, the SERS signals were ~12.9 and 18.6× greater than that of the AuNP, respectively. The results on LFA strips confirmed that the 56-nm Ag@AuNS provide the highest SERS signal in the LFA and was, therefore, selected as the present inventors' optimized SERS-LFA nanomaterial.

### Determination of SERS-LFA Sensitivity

A recent publication has reported an antibody-based LFA using AuNP for colorimetric detection P. gingivalis LPS with LOD of ~20 ng/ml in aqueous samples and ~45 ng/mL in human saliva samples [31]. Building on those results, the goal of this work is to produce a SERS-LFA with clinically relevant limits of detection (LOD). In clinical settings, healthy individuals present ~10 ng/ml concentrations of LPS from all bacteria types, whereas individuals with periodontitis will have LPS concentrations an order of magnitude higher [65]. Thus, for clinical relevance, the SERS-LFA must have a limit of detection of ~10 ng/ml. To compare LODs, the present inventors performed a calibration for the conventional LFA with colorimetric detection and the SERS-LFA using 56 nm Ag@AuNS. The present inventors specifically compare the developed SERS-LFA with a AuNP-based method rather than AuNS, because AuNP are optimized for colorimetric detection and have a superior absorbance in the visible range.

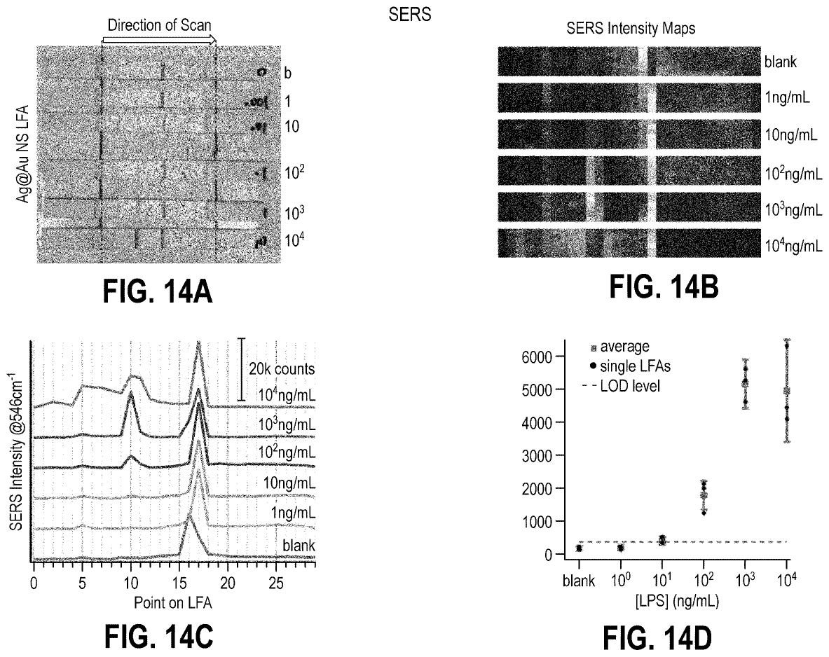

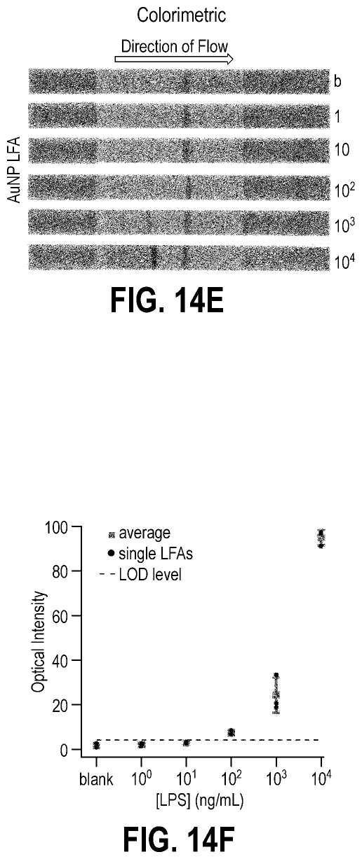

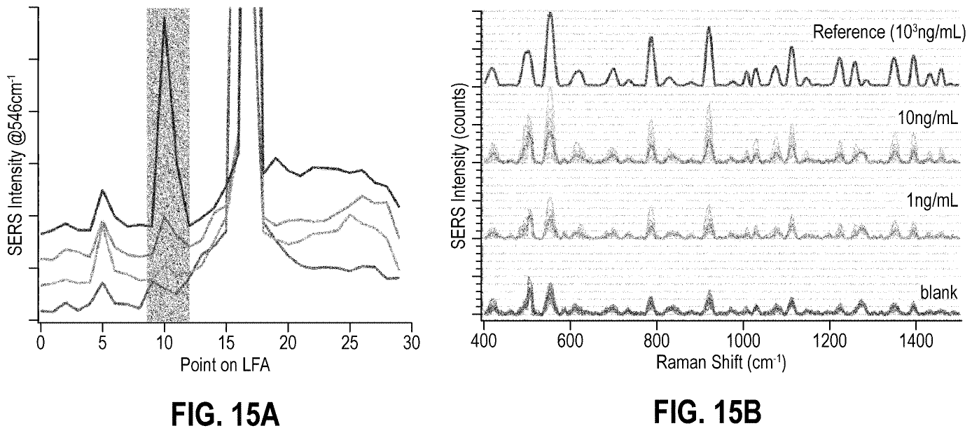

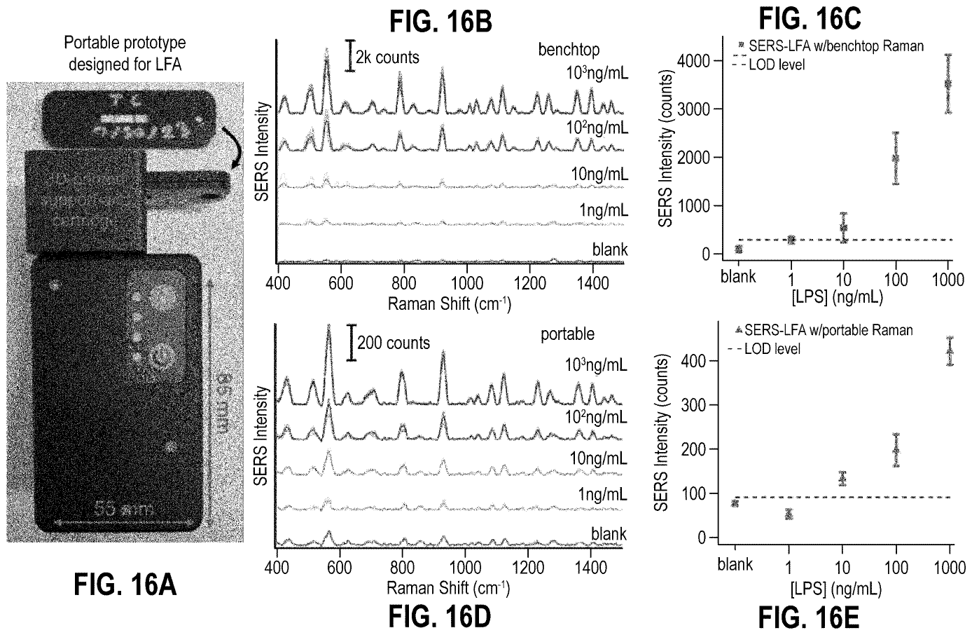

FIG. 14A-14F shows the comparison between calibrations performed on SERS-LFA and colorimetric-LFA. The Ag@AuNS LFA (FIG. 14A) were tested scanning the laser across the LFA long-axis (30×1-mm steps) 6 times and averaging the results (FIG. 7). FIG. 14A-14F reports each line-scan as a 6×30 colormap (FIG. 14B) and the average line-scan results (FIG. 14C) for the selected reporter peak (549 cm⁻¹). The test line intensity (at point #10) as a function of LPS concentration is reported in FIG. 14D. FIG. 14E includes equivalent photographs of the AuNP LFAs and the analyzed optical intensity as a function of LPS concentration using ImageJ. In agreement with previous results, the colorimetric LFA analysis can detect LPS at concentrations above 10 ng/ml, with the first concentration observed above the LOD in this test at 100 ng/mL. However, the SERS-LFA results show a detection limit below 10 ng/ml, with the first observable concentration at 10 ng/ml (FIG. 14D). These results demonstrate ~10× improvement offered by the SERS detection mechanism, bringing the LOD within the clinically relevant range for targeted PG LPS. Interestingly, while the LOD is between 10 and 1 ng/mL, the line scans (FIG. 14C) show the reporter peak monotonically increasing for the test line starting at 1 ng/mL. FIG. 15A shows an expanded version of FIG. 14C to observe the target-line peak at low concentration. The present inventors believe that the LOD value was influenced by non-specific binding in the blank strip due to the low stability of NS after full functionalization (i.e., reporter+antibody), as it can be appreciated from the off-line variance observed in the maps (FIG. 14B). The present inventors believe that with further optimization of the functionalization and assay process in this specific application, LOD below 1 ng/ml can be achieved. Finally, the present inventors' results show an improved LOD compared to the colorimetric method but also a significant improvement over simple test-line visualization (common readout method for LFA), which can be observed only above 100 ng/ml (FIG. 14E). FIG. 15B shows the average as well as individual spectra for each measurement (n=18; 6 lines×3 sets) on the test line. As it can be observed, while the average agrees with the results shown reported in FIG. 16D (no significant difference between blank and 1 ng/ml), it is clearly noticeable that the number of high-intensity spectra in 1 ng/mL is higher than for the blank (FIG. 15B). This result suggests that there is likely an observable significant difference even for 1 ng/mL samples; however, conventional average of spectral signal may not be the ideal analysis to observe this difference. This observation agrees with recent studies on optimal sampling of SERS-LFA, where counting the number of SERS particles rather the average signal was used to build calibrations [75].

Testing with Portable Raman System

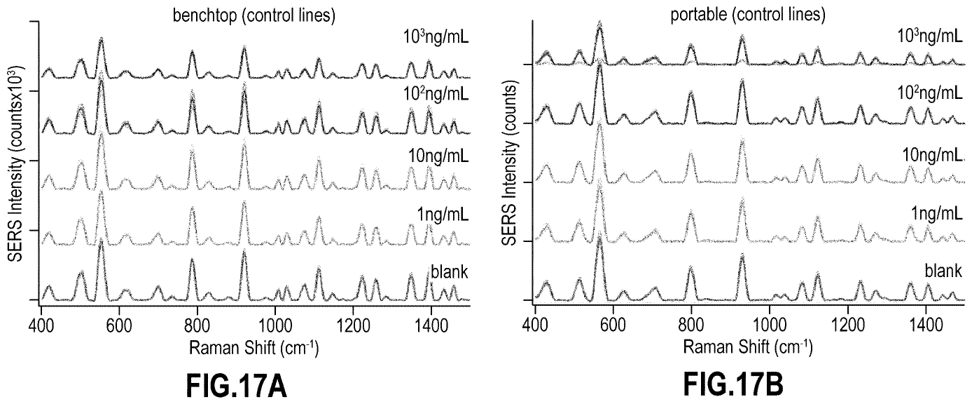

The SERS-LFA designed and optimized in this work aims to provide a high-sensitivity option in POC applications. One of the hurdles in the translation of SERS-LFA to the POC domain is the complexity, size and cost of Raman systems that typically contain microscopes, benchtop laser and spectrometer systems. To enable this paradigm shift, Raman systems have been developed to be handheld and optimized for specific POC applications. For SERS-LFA, small systems with a test/control line-wide laser excitation are ideal. This type of system has been previously tested with SERS-LFA showing clinically significant results [60, 61]. Herein, the present inventors tested a ‘hand-held’ SERS-LFA scanner prototype from Wasatch Photonics designed for LFA-based POC applications. This system is battery operated, portable (85×55×30 mm) and the laser excitation has been designed to form a line shape, to maximize signal from typical LFA strips. The prototype was provided with a 3D-printed support and cartridge for easy reading of LFA strips (see photos in FIG. 16A). The cartridge fits in the support placing the LFA test and control lines and pre-aligned in the line-focus of the laser. To demonstrate the possible translation of the developed SERS-LFA to the POC, the present inventors compared the results obtained with the benchtop Raman system to results obtained with the portable device on a set of LFA with several target LPS concentrations. FIGS. 16A-16E report the spectra obtained with the two systems and the average results for each concentration. The results obtained with the prototype designed for POC settings are in agreement (LOD and sensitivity) with those obtained using the benchtop system. Both systems have an LOD <10 ng/mL, which was the present inventors' target to make the LOD of this test clinically relevant. While spectra differ in intensity and resolution due to the difference in detector size and temperature used in the two systems (10° C. for benchtop and uncooled for portable), the trends of SERS intensity as a function of concentration are remarkably similar. FIGS. 17A and 17B report the spectra for the control line, which agrees with the conclusions from the test-line results of FIGS. 16A-16E. Interestingly, the SERS measurements on the benchtop system show a somewhat greater variance than for the portable system. This difference is likely due to the measurements methodology. In the benchtop system the test/control line is scanned horizontally at multiple vertical locations, while in the portable device the entire line is measured at one time. Furthermore, in the portable system the excitation position is manually adjusted to optimize the signal from the test/control line, resulting in a reduced variance in the measurements. Overall, the results indicate that there is significant variability along and across the line and, therefore, the whole-line excitation solution is ideal for SERS-LFA to reduce variability.

### CONCLUSION

In summary, the present inventors have demonstrated the use of an optimized SERS-LFA to improve the analytical performance for the detection of P. gingivalis, an important bacterium deleterious to oral health. To this end, the present inventors characterized four lab-produced nanomaterials and compared their SERS performance to commercially available AuNP. Through this characterization the present inventors determined that 56 nm Ag@AuNS were the optimal material for use in SERS-LFAs. The present inventors compared the sensitivity of the optimized LFA to that of standard optical (colorimetric) LFAs and demonstrated an improvement in the LFA sensitivity that brings the detection limit of SERS-LFA below 10 ng/mL, within the clinically relevant concentration window. The present inventors' work represents the first study to use Ag@AuNS for SERS-LFA measurements, providing exceptional SERS signal, and to compare more than two nanomaterials for application in SERS-LFAs. Additionally, the use of an automated scanner (collection time 5 min) and a portable spectrometer (collection time <1 min) shows that these tests could be administered and measured in clinical settings in under 15 min. Overall, the present inventors have demonstrated a new application for SERS-LFA, while also reporting for the first time a series of nanomaterials in these systems. By incorporating both optimized nanomaterials and portable measurements, the potential application of this technology can greatly improve the quality of clinical diagnostics at the point of care.

### REFERENCES

1. S. R. Lyons, A. L. Griffen and E. J. Leys, J. Clin. Microbiol., 2000, 38, 2362-2365.
2. A. J. Steckl and P. Ray, ACS Sens., 2018, 3, 2025-2044.
3. S. Dalirirad, D. Han and A. J. Steckl, ACS Omega, 2020, 5, 32890-32898.
4. W. Hao, Y. Huang, L. Wang, J. Liang, S. Yang, L. Su and X. Zhang, ACS Appl. Mater. Interfaces, 2023, 15, 9665-9674.
5. E. B. Aydin, M. Aydin and M. K. Sezgintürk, Adv. Clin. Chem., 2023, 113, 1-41.
6. T. W. Pittman, D. B. Decsi, C. Punyadeera and C. S. Henry, Theranostics, 2023, 13, 1091-1108.
7. C. R. H. Raetz and C. Whitfield, Annu. Rev. Biochem., 2002, 71, 635-700.
8. S. Harm, C. Schildböck, K. Strobl and J. Hartmann, Sci. Rep., 2021, 11, 4192.
9. S. H. Rhee, Intest. Res., 2014, 12, 90-95.
10. G. Klein and S. Raina, Int. J. Mol. Sci., 2019, 20, 356.
11. B. S. Park and J.-O. Lee, Exp. Mol. Med., 2013, 45, e66.
12. J. Mysak, S. Podzimek, P. Sommerova, Y. Lyuya-Mi, J. Bartova, T. Janatova, J. Prochazkova and J. Duskova, J. Immunol. Res., 2014, 2014, 476068.
13. M. Klukowska, J. C. Haught, S. Xie, B. Circello, C. S. Tansky, D. Khambe, T. Huggins and D. J. White, J. Clin. Dent., 2017, 28, 16-26.
14. S. Xie, C. Haught, C. Tansky, M. Klukowska, P. Hu, D. Ramsey, B. Circello, T. Huggins and D. White, Am. J. Dent., 2018, 31, 215-224.
15. P.-L. Wang and K. Ohura, Crit. Rev. Oral Biol. Med., 2002, 13, 132-142.
16. J. L. C. Mougeot, C. B. Stevens, B. J. Paster, M. T. Brennan, P. B. Lockhart and F. K. B. Mougeot, J. Oral Microbiol., 2017, 9, 1281562.
17. M. J. Gui, S. G. Dashper, N. Slakeski, Y. Y. Chen and E. C. Reynolds, Mol. Oral Microbiol., 2016, 31, 365-378.
18. B. Rodríguez-Lozano, J. González-Febles, J. L. Garnier-Rodríguez, S. Dadlani, S. Bustabad-Reyes, M. Sanz, F. Sánchez-Alonso, C. Sánchez-Piedra, E. González-Dávila and F. Díaz-González, Arthritis Res. Ther., 2019, 21, 27.
19. C.-K. Chen, Y.-T. Wu and Y.-C. Chang, Alzheimer's Res. Ther., 2017, 9, 56.
20. S. Poole, S. K. Singhrao, L. Kesavalu, M. A. Curtis and S. Crean, J. Alzheimer's Dis., 2013, 36, 665-677.
21. S. S. Dominy, C. Lynch, F. Ermini, M. Benedyk, A. Marczyk, A. Konradi, M. Nguyen, U. Haditsch, D. Raha, C. Griffin, L. J. Holsinger, S. Arastu-Kapur, S. Kaba, A. Lee, M. I. Ryder, B. Potempa, P. Mydel, A. Hellvard, K. Adamowicz, H. Hasturk, G. D. Walker, E. C. Reynolds, R. L. M. Faull, M. A. Curtis, M. Dragunow and J. Potempa, Sci. Adv., 2019, 5, eaau3333.
22. W. He, M. You, W. Wan, F. Xu, F. Li and A. Li, Trends Biotechnol., 2018, 36, 1127-1144.
23. Y. C. Chow, H. C. Yam, B. Gunasekaran, W. Y. Lai, W. Y. Wo, T. Agarwal, Y. Y. Ong, S. L. Cheong and S.-A. Tan, Front. Cell. Infect. Microbiol., 2022, 12, 987683.
24. H. Tamura, J. Reich and I. Nagaoka, Biomedicines, 2021, 9, 536.
25. K. Reither, E. Saathoff, J. Jung, L. T. Minja, I. Kroidl, E. Saad, J. F. Huggett, E. N. Ntinginya, L. Maganga, L. Maboko and M. Hoelscher, BMC Infect. Dis., 2009, 9, 141.
26. M. Cho, L. Chun, M. Lin, W. Choe, J. Nam and Y. Lee, Sens. Actuators, B, 2012, 174, 490-494.
27. S. E. Kim, W. Su, M. Cho, Y. Lee and W. S. Choe, Anal. Biochem., 2012, 424, 12-20.
28. R. S. Loreen, M. M. Heather and M. Harshini, Detection Methods for Lipopolysaccharides: Past and Present, IntechOpen, Rijeka, 2017.
29. Khelifa, L.; Hu, Y.; Jiang, N.; Yetisen, A. K., Lateral flow assays for hormone detection. Lab on a Chip 2022, 22 (13), 2451-2475.
30. Kuswandi, B.; Hidayat, M. A.; Noviana, E., Paper-based sensors for rapid important biomarkers detection. Biosensors and Bioelectronics: X 2022, 12, 100246.
31. Han, D.; Xie, S.; Steckl, A. J., Salivary endotoxin detection using combined mono/polyclonal antibody-based sandwich-type lateral flow immunoassay device. Sensors & Diagnostics 2023, 2 (6), 1460-1468.
32. Dalirirad, S.; Steckl, A. J., Lateral flow assay using aptamer-based sensing for on-site detection of dopamine in urine. Analytical Biochemistry 2020, 596, 113637.
33. Dalirirad, S.; Steckl, A. J., Aptamer-based lateral flow assay for point of care cortisol detection in sweat. Sensors and Actuators B: Chemical 2019, 283, 79-86.
34. Liu, Y.; Zhan, L.; Qin, Z.; Sackrison, J.; Bischof, J. C., Ultrasensitive and Highly Specific Lateral Flow Assays for Point-of-Care Diagnosis. ACS Nano 2021, 15 (3), 3593-3611.
35. Budd, J.; Miller, B. S.; Weckman, N. E.; Cherkaoui, D.; Huang, D.; Decruz, A. T.; Fongwen, N.; Han, G.-R.; Broto, M.; Estcourt, C. S.; Gibbs, J.; Pillay, D.; Sonnenberg, P.; Meurant, R.; Thomas, M. R.; Keegan, N.; Stevens, M. M.; Nastouli, E.; Topol, E. J.; Johnson, A. M.; Shahmanesh, M.; Ozcan, A.; Collins, J. J.; Fernandez Suarez, M.; Rodriguez, B.; Peeling, R. W.; McKendry, R. A., Lateral flow test engineering and lessons learned from COVID-19. Nature Reviews Bioengineering 2023, 1 (1), 1331.
36. Frantz, E.; Li, H.; Steckl, A. J., Quantitative hematocrit measurement of whole blood in a point-of-care lateral flow device using a smartphone flow tracking app. Biosens Bioelectron 2020, 163, 112300.
37. Jain, S.; S. Dandy, D.; J. Geiss, B.; S. Henry, C., Padlock probe-based rolling circle amplification lateral flow assay for point-of-need nucleic acid detection. Analyst 2021, 146 (13), 4340-4347.
38. Wang, Y.; Qin, Z.; Boulware, D. R.; Pritt, B. S.; Sloan, L. M.; Gonzalez, I. J.; Bell, D.; Rees-Channer, R. R.; Chiodini, P.; Chan, W. C. W.; Bischof, J. C., Thermal Contrast Amplification Reader Yielding 8-Fold Analytical Improvement for Disease Detection with Lateral Flow Assays. Analytical Chemistry 2016, 88 (23), 11774-11782.
39. Kim, K.; Kashefi-Kheyrabadi, L.; Joung, Y.; Kim, K.; Dang, H.; Chavan, S. G.; Lee, M.-H.; Choo, J., Recent advances in sensitive surface-enhanced Raman scattering-based lateral flow assay platforms for point-of-care diagnostics of infectious diseases. Sensors and Actuators B: Chemical 2021, 329, 129214.
40. Mulvaney, S. P.; Kidwell, D. A.; Lanese, J. N.; Lopez, R. P.; Sumera, M. E.; Wei, E., Catalytic lateral flow immunoassays (cLFIATm): Amplified signal in a self-contained assay format. Sensing and Bio-Sensing Research 2020, 30, 100390.
41. Hwang, J.; Lee, S.; Choo, J., Application of a SERS-based lateral flow immunoassay strip for the rapid and sensitive detection of staphylococcal enterotoxin B. Nanoscale 2016, 8 (22), 11418-11425.
42. Cao, X.; Song, Q.; Sun, Y.; Mao, Y.; Lu, W.; Li, L., A SERS-LFA biosensor combined with aptamer recognition for simultaneous detection of thrombin and PDGF-BB in prostate cancer plasma. Nanotechnology 2021, 32 (44), 445101.
43. Shi, Q.; Huang, J.; Sun, Y.; Deng, R.; Teng, M.; Li, Q.; Yang, Y.; Hu, X.; Zhang, Z.; Zhang, G., A SERS-based multiple immuno-nanoprobe for ultrasensitive detection of neomycin and quinolone antibiotics via a lateral flow assay. Microchim Acta 2018, 185 (2), 84.
44. Li, J.; Xu, J.; Pan, Y.; Zhu, Y.; Wang, Y.; Chen, S.; Wei, X., Au@Ag-labeled SERS lateral flow assay for highly sensitive detection of allergens in milk. Food Science and Human Wellness 2023, 12 (3), 912-919.
45. Wang, C.; Wang, C.; Li, J.; Tu, Z.; Gu, B.; Wang, S., Ultrasensitive and multiplex detection of four pathogenic bacteria on a bi-channel lateral flow immunoassay strip with three-dimensional membrane-like SERS nanostickers. Biosensors and Bioelectronics 2022, 214, 114525.
46. Srivastav, S.; Dankov, A.; Adanalic, M.; Grzeschik, R.; Tran, V.; Pagel-Wieder, S.; Gessler, F.; Spreitzer, I.; Scholz, T.; Schnierle, B.; Anastasiou, 0. E.; Dittmer, U.; Schlucker, S., Rapid and Sensitive SERS-Based Lateral Flow Test for SARS-COV2-Specific IgM/IgG Antibodies. Analytical Chemistry 2021, 93 (36), 12391-12399.
47. A. Hassanain, W.; Spoors, J.; L. Johnson, C.; Faulds, K.; Keegan, N.; Graham, D., Rapid ultra-sensitive diagnosis of Clostridium difficile infection using a SERS-based lateral flow assay. Analyst 2021, 146 (14), 4495-4505.
48. Mao, Y.; Sun, Y.; Xue, J.; Lu, W.; Cao, X., Ultra-sensitive and high efficiency detection of multiple non-small cell lung cancer-related miRNAs on a single test line in catalytic hairpin assembly-based SERS-LFA strip. Analytica Chimica Acta 2021, 1178, 338800.
49. Yan, S.; Liu, C.; Fang, S.; Ma, J.; Qiu, J.; Xu, D.; Li, L.; Yu, J.; Li, D.; Liu, Q., SERS-based lateral flow assay combined with machine learning for highly sensitive quantitative analysis of Escherichia coli 0157: H7. Anal Bioanal Chem 2020, 412 (28), 7881-7890.
50. Wang, R.; Kim, K.; Choi, N.; Wang, X.; Lee, J.; Jeon, J. H.; Rhie, G.-e.; Choo, J., Highly sensitive detection of high-risk bacterial pathogens using SERS-based lateral flow assay strips. Sensors and Actuators B: Chemical 2018, 270, 72-79.
51. Li, Z.; Gu, Y.; Ge, S.; Mao, Y.; Gu, Y.; Cao, X.; Lu, D., An aptamer-based SERS-LFA biosensor with multiple channels for the ultrasensitive simultaneous detection of serum VEGF and osteopontin in cervical cancer patients. New J. Chem. 2022, 46 (43), 20629-20642.
52. Zhan, Y.; Fei, R.; Lu, Y.; Wan, Y.; Wu, X.; Dong, J.; Meng, D.; Ge, Q.; Zhao, X., Ultrasensitive detection of multiple Alzheimer's disease biomarkers by SERS-LFA. Analyst 2022, 147 (18), 41244131.
53. Liu, X.; Yang, X.; Li, K.; Liu, H.; Xiao, R.; Wang, W.; Wang, C.; Wang, S., Fe304@Au SERS tags-based lateral flow assay for simultaneous detection of serum amyloid A and C-reactive protein in unprocessed blood sample. Sensors and Actuators B: Chemical 2020, 320, 128350.
54. Lin, L.-K.; Stanciu, L. A., Bisphenol A detection using gold nanostars in a SERS improved lateral flow immunochromatographic assay. Sensors and Actuators B: Chemical 2018, 276, 222-229.
55. Zhang, D.; Huang, L.; Liu, B.; Ni, H.; Sun, L.; Su, E.; Chen, H.; Gu, Z.; Zhao, X., Quantitative and ultrasensitive detection of multiplex cardiac biomarkers in lateral flow assay with core-shell SERS nanotags. Biosens Bioelectron 2018, 106, 204-211.
56. Hassanain, W. A.; Spoors, J.; Johnson, C. L.; Faulds, K.; Keegan, N.; Graham, D., Rapid ultra-sensitive diagnosis of Clostridium difficile infection using a SERS-based lateral flow assay. Analyst 2021, 146 (14), 4495-4505.
57. Wu, Z., Simultaneous Detection of Listeria monocytogenesandSalmonella typhimuriumby a SERS-Based Lateral Flow Immunochromatographic Assay. Food Analytical Methods 2019, 12 (5), 10861091.
58. Wang, X.; Choi, N.; Cheng, Z.; Ko, J.; Chen, L.; Choo, J., Simultaneous detection of dual nucleic acids using a SERS-based lateral flow assay biosensor. Analytical Chemistry 2017, 89 (2), 11631169.
59. Sanchez-Purra, M.; Carre-Camps, M.; De Puig, H.; Bosch, I.; Gehrke, L.; Hamad-Schifferli, K., Surface-Enhanced Raman Spectroscopy-Based Sandwich Immunoassays for Multiplexed Detection of Zika and Dengue Viral Biomarkers. ACS Infectious Diseases 2017, 3 (10), 767-776.
60. Tran, V.; Walkenfort, B.; Konig, M.; Salehi, M.; Schlucker, S., Rapid, Quantitative, and Ultrasensitive Point-of-Care Testing: A Portable SERS Reader for Lateral Flow Assays in Clinical Chemistry. Angew Chem Int Ed Engl 2019, 58 (2), 442-446.
61. 33. Soliman, C.; Faircloth, J.; Tu, D.; Mabbott, S.; Maitland, K.; Cote, G., Exploring the Clinical Utility of Raman Spectroscopy for Point-of-Care Cardiovascular Disease Biomarker Detection. Applied Spectroscopy 2023, 77 (10), 1181-1193.
62. Y. Ji, Y. Huang, Z. Cheng, W. Hao, G. Liu, Y. Liu and X. Zhang, J. Agric. Food Chem., 2023, 71, 10250-10268.
63. Corredor, Z.; Suarez-Molina, A.; Fong, C.; Cifuentes-C, L.; Guauque-Olarte, S., Presence of periodontal pathogenic bacteria in blood of patients with coronary artery disease. Scientific Reports 2022, 12, 1241.
64. Dominy, S. S.; Lynch, C.; Ermini, F.; Benedyk, M.; Marczyn, A.; Konradi, A.; Nguyen, M.; Haditsch, U.; Raha, D.; Griffin, C.; Holsinger, L. J.; Arastu-Kapur, S.; Kaba, S.; Lee, A.; Ryder, M. I.; Potempa, B.; Mydel, P.; Hellvard, A.; Adamowicz, K.; Hasturk, H.; Walker, G. D.; Reynolds, E. C.; Faull, R. L. M.; Curtis, M. A.; Dragunow, M.; Potempa, J., Porphyromonas gingivalis in Alzheimer's disease brains: Evidence for disease causation and treatment with small-molecule inhibitors. Science Advances 2019 5 (1), eaau3333.
65. Zaric, S.; Strachan, A.; Kurushima, Y.; Dong, A.; McIlwaine, C.; Harrington, Z.; Nibali, L.; Foey, A.; Ide, M., Evaluating clinical utility of subgingival and salivary endotoxin activity levels as periodontal biomarkers. Frontiers in Oral Health 2022, 3.
66. Yuan, H.; Fales, A. M.; Khoury, C. G.; Liu, J.; Vo-Dinh, T., Spectral Characterization and Intracellular Detection of Surface-Enhanced Raman Scattering (SERS)-Encoded Plasmonic Gold Nanostars. J Raman Spectrosc 2013, 44 (2), 234-239.
67. J. Zhu, W. Li, M. Zhu, W. Zhang, W. Niu and G. Liu, AIP Adv., 2014, 4, 031338.
68. D. A. Armbruster and T. Pry, Clin. Biochem. Rev., 2008, 29, S49-S52.
69. O. Deutsch, Y. Fleissig, B. Zaks, G. Krief, D. J. Aframian and A. Palmon, Electrophoresis, 2008, 29, 4150-4157.
70. Turkevich, J.; Stevenson, P. C.; Hillier, J., A study of the nucleation and growth processes in the synthesis of colloidal gold. Discuss. Faraday Soc. 1951, 11, 55.
71. Fales, A. M.; Yuan, H.; Vo-Dinh, T., Development of Hybrid Silver-Coated Gold Nanostars for Nonaggregated Surface-Enhanced Raman Scattering. J. Phys. Chem. C 2014, 118 (7), 3708-3715.
72. Schindelin, J.; Arganda-Carreras, I.; Frise, E.; Kaynig, V.; Longair, M.; Pietzsch, T.; Preibisch, S.; Rueden, C.; Saalfeld, S.; Schmid, B.; Tinevez, J. Y.; White, D. J.; Hartenstein, V.; Eliceiri, K.; Tomancak, P.; Cardona, A., Fiji: an open-source platform for biological-image analysis. Nat Methods 2012, 9 (7), 676-82.
73. Sajanlal, P. R.; Sreeprasad, T. S.; Samal, A. K.; Pradeep, T., Anisotropic nanomaterials: structure, growth, assembly, and functions. Nano Rev 2011, 2.
74. Vang, D.; Strobbia, P., Analysis of Nanostar Reshaping Kinetics for Optimal Substrate Fabrication. Applied Spectroscopy 2023, 77 (3), 270-280.
75. Tuckmantel Bido, A.; Brolo, A. G., Digital SERS Protocol Using Au Nanoparticle-Based Extrinsic Raman Labels for the Determination of SARS-COV-2 Spike Protein in Saliva Samples. ACS Applied Nano Materials 2023, 6 (17), 15426-15436.

Although not described in detail herein, other steps which are readily interpreted from or incorporated along with the disclosed embodiments shall be included as part of the invention. The embodiments that have been described herein provide specific examples to portray inventive elements, but will not necessarily cover all possible embodiments commonly known to those skilled in the art.

What is claimed is:

1. A lateral flow assay device for detecting the presence of, or quantifying an amount or concentration of, lipopolysaccharide in a sample, the device comprising:
a conjugate region including a binding reagent coupled to a signal-generating particle, wherein the binding reagent comprises a monoclonal antibody configured to bind lipopolysaccharide, and wherein the binding reagent is mobile relative to the device; and
an analytical membrane in fluid communication with the conjugate region, the analytical membrane comprising a test region and a control region,
(i) wherein the test region includes an immobilized capture reagent configured to capture a complex including the lipopolysaccharide and the binding reagent, wherein the immobilized capture reagent comprises a polyclonal antibody, and
(ii) wherein the control region comprises a control component configured to capture the binding reagent coupled to the signal-generating particle.

2. The device of claim 1, wherein the analytical membrane comprises nitrocellulose.

3. The device of claim 2, wherein the analytical membrane comprises a porous nitrocellulose membrane.

4. The device of claim 1, wherein the sample comprises saliva.

5. The device of claim 4, wherein the saliva is human saliva.

6. The device of claim 1, wherein the signal-generating particle comprises a metallic particle, a dyed particle, a fluorescent particle, a magnetic particle, or a combination thereof.

7. The device of claim 1, wherein the signal-generating particle comprises a nanoparticle.

8. The device of claim 7, wherein the nanoparticle comprises a gold nanoparticle.

9. The device of claim 1, wherein the control region is disposed downstream of the test region.

10. The device of claim 1, wherein the device is configured to provide a visually detectable signal at the test region.

11. The device of claim 1, wherein the device is configured for qualitative detection of lipopolysaccharide.

12. The device of claim 1, wherein the device is configured for quantitative determination of lipopolysaccharide.

13. The device of claim 1, wherein detection of lipopolysaccharide is indicative of the presence and/or amount of bacterial endotoxin in the sample.

14. The device of claim 1, wherein the conjugate region comprises a porous conjugate pad containing a dried conjugate formulation.

15. The device of claim 14, wherein the dried conjugate formulation further comprises one or more stabilizers selected from sugars, proteins, surfactants, polymers, and preservatives.

16. The device of claim 1, wherein the signal-generating particle is a SERS-active particle and is associated with a Raman reporter.

17. The device of claim 16, wherein the device is configured to generate a Raman-detectable signal at the test region.

18. The device of claim 16, wherein the SERS-active particle is chosen from a gold nanoparticle, a gold nanostar, a silver-coated gold nanostar, and a combination thereof.

19. The device of claim 18, wherein the SERS-active particle comprises a silver-coated gold nanostar.

20. The device of claim 19, wherein the silver-coated gold nanostar has an average size of about 56 nm.

21. The device of claim 16, wherein the device is configured for Raman spectroscopic detection of lipopolysaccharide at a concentration below about 10 ng/mL.

22. The device of claim 1, wherein the control component comprises protein G.

23. The device of claim 1, further comprising a sample receiving region in fluid communication with the conjugate region.

24. The device of claim 1, further comprising an absorbent region in fluid communication with the analytical membrane.

25. A lateral flow assay device for detecting the presence of, or quantifying an amount or concentration of, lipopolysaccharide in a sample, the device comprising:
a conjugate region including a binding reagent coupled to a signal-generating particle, wherein the binding reagent comprises a first polyclonal antibody configured to bind lipopolysaccharide, and wherein the binding reagent is mobile relative to the device;
an analytical membrane in fluid communication with the conjugate region, the analytical membrane comprising a test region and a control region,
(i) wherein the test region includes an immobilized capture reagent configured to capture a complex including the lipopolysaccharide and the binding reagent, wherein the immobilized capture reagent comprises a second polyclonal antibody, and
(ii) wherein the control region comprises a control component configured to capture the binding reagent coupled to the signal-generating particle.

26. The device of claim 25, wherein the analytical membrane comprises nitrocellulose.

27. The device of claim 26, wherein the analytical membrane comprises a porous nitrocellulose membrane.

28. The device of claim 25, wherein the sample comprises saliva.

29. The device of claim 28, wherein the saliva is human saliva.

30. The device of claim 25, wherein the signal-generating particle comprises a metallic particle, a dyed particle, a fluorescent particle, a magnetic particle, or a combination thereof.

31. The device of claim 25, wherein the signal-generating particle comprises a nanoparticle.

32. The device of claim 31, wherein the nanoparticle comprises a gold nanoparticle.

33. The device of claim 25, wherein the control region is disposed downstream of the test region.

34. The device of claim 25, wherein the device is configured to provide a visually detectable signal at the test region.

35. The device of claim 25, wherein the device is configured for qualitative detection of lipopolysaccharide.

36. The device of claim 25, wherein the device is configured for quantitative determination of lipopolysaccharide.

37. The device of claim 25, wherein detection of lipopolysaccharide is indicative of the presence and/or amount of bacterial endotoxin in the sample.

38. The device of claim 25, wherein the conjugate region comprises a porous conjugate pad containing a dried conjugate formulation.

39. The device of claim 38, wherein the dried conjugate formulation further comprises one or more stabilizers selected from sugars, proteins, surfactants, polymers, and preservatives.

40. The device of claim 25, wherein the signal-generating particle is a SERS-active particle and is associated with a Raman reporter.

41. The device of claim 40, wherein the device is configured to generate a Raman-detectable signal at the test region.

42. The device of claim 40, wherein the SERS-active particle is chosen from a gold nanoparticle, a gold nanostar, a silver-coated gold nanostar, and a combination thereof.

43. The device of claim 41, wherein the SERS-active particle comprises a silver-coated gold nanostar.

44. The device of claim 43, wherein the silver-coated gold nanostar has an average size of about 56 nm.

45. The device of claim 25, wherein the device is configured for Raman spectroscopic detection of lipopolysaccharide at a concentration below about 10 ng/mL.

46. The device of claim 25, wherein the control component comprises protein G.

47. The device of claim 25, further comprising a sample receiving region in fluid communication with the conjugate region.

48. The device of claim 25, further comprising an absorbent region in fluid communication with the analytical membrane.

49. A lateral flow assay device for determining the presence of, or quantifying an amount or concentration of, analyte in a sample, the device comprising:
a blocking pad comprising a binding reagent coupled to a SERS-active particle and associated Raman reporter, wherein the binding reagent is configured to bind an analyte, and wherein the SERS-active particle comprises a silver-coated gold nanostar; and
an analytical membrane in fluid communication with the conjugate region, the analytical membrane comprising a test region and a control region, wherein the test region comprises an immobilized capture reagent configured to capture a complex comprising the analyte and the binding reagent, and wherein the control region comprises a control component configured to capture the binding reagent coupled to the SERS-active particle;
wherein the device is configured to generate a Raman-detectable signal at the test region.

50. The device of claim 49, wherein the analytical membrane comprises nitrocellulose.

51. The device of claim 49, wherein the analytical membrane comprises a porous nitrocellulose membrane.

52. The device of claim 49, wherein the blocking pad comprises fiberglass.

53. The device of claim 49, further comprising one or more blocking agents disposed on and/or in the blocking pad.

54. The device of claim 49, wherein the sample comprises saliva.

55. The device of claim 54, wherein the saliva is human saliva.

56. The device of claim 49, wherein the binding reagent comprises an antibody.

57. The device of claim 56, wherein the antibody is selected from a monoclonal antibody and a polyclonal antibody.

58. The device of claim 49, wherein the immobilized capture reagent comprises a polyclonal antibody.

59. The device of claim 49, wherein the silver-coated gold nanostar has an average size of about 56 nm.

60. The device of claim 49, wherein the binding reagent comprises a monoclonal antibody and the immobilized capture reagent comprises a polyclonal antibody.

61. The device of claim 60, wherein the binding reagent comprises a monoclonal antibody conjugated to the SERS-active particle.

62. The device of claim 49, wherein the binding reagent comprises a polyclonal antibody and the immobilized capture reagent comprises a polyclonal antibody.

63. The device of claim 62, wherein the binding reagent comprises a polyclonal antibody conjugated to the SERS-active particle.

64. The device of claim 49, wherein the device is configured for Raman spectroscopic detection of lipopolysaccharide at a concentration below about 10 ng/mL.

65. The device of claim 49, wherein detection of lipopolysaccharide is indicative of the presence and/or amount of bacterial endotoxin in the sample.

66. The device of claim 49, wherein the control component comprises protein G.

67. The device of claim 49, further comprising a sample receiving region in fluid communication with the conjugate region.

68. The device of claim 49, further comprising an absorbent region in fluid communication with the analytical membrane.

69. A combination comprising the device of claim 49 and a Raman scanner.

70. The combination of claim 69, wherein the Raman scanner is configured to measure a SERS signal from the SERS-active particle along the lateral flow direction of the device with a selected resolution.

71. The combination of claim 70, wherein the SERS signal is generated via laser excitation.

72. The combination of claim 71, wherein the laser is focused on the test region and/or control region of the device.

73. The combination of claim 72, wherein the excitation position of the laser can be adjusted to optimize the signal from the test region and/or control region.
